(19) United States
(12) Patent Application Publication
(10) Pub. No.: US 2026/0292026 A1
(43) Pub. Date: Sep. 24, 2026

(54) MEDICAL-DEVICE LORA COMMUNICATION SYSTEM WITH PROTECTED CLINICAL-CONTEXT METADATA, AUTHORIZED GATEWAY VALIDATION, AND DOWNSTREAM CLINICAL RELEASE CONTROL

(71) Applicant: OneSource Solutions International, Inc., Sudbury, MA (US)

(72) Inventors: Harold Arkoff, Sudbury, MA (US); Vedran Jukic, Trieste (IT)

(73) Assignee: OneSource Solutions International, Inc., Sudbury, MA (US)

(21) Appl. No.: 19/680,687

(22) Filed: May 18, 2026

Related U.S. Application Data
(63) Continuation-in-part of application No. 19/053,381, filed on Feb. 13, 2025, now Pat. No. 12,651,667.

(51) Int. Cl. H04L 67/12; H04L 9/40

(52) U.S. Cl. CPC H04L 67/12; H04L 63/0428

(57) ABSTRACT

A medical-device LoRa communication system includes a LoRa communication module coupled to a host medical device or patient-associated medical device. The module obtains medical-device, physiological, alarm, device-status, or device-identity data and generates an encrypted LoRa packet having a relay-readable header, protected clinical-context metadata, and an encrypted payload. One or more LoRa relay nodes forward the packet toward an authorized gateway using the relay-readable header without decrypting the payload or accessing the protected clinical-context metadata in readable form. The authorized gateway validates packet information against authorization records and patient-context or device-context records before downstream clinical release. When validation fails, downstream clinical routing is prevented. When validation succeeds, the gateway decrypts the payload, binds the data to a validated context record, and makes the data available to an authorized electronic medical record system, medical device data system, medical data governance system, or other downstream system.

### CROSS-REFERENCE TO RELATED APPLICATIONS

This application is a continuation-in-part of U.S. Patent Application No. 19/053,381, filed Feb. 13, 2025, titled “Wearable Medical Device Data Connectivity System Using LoRa,” which is a continuation-in-part of U.S. Patent Application No. 18/581,344, filed Feb. 19, 2024, titled “Wearable Medical Device Data Connectivity System Using LoRa.” The contents of U.S. Patent Application Nos. 19/053,381 and 18/581,344 are incorporated herein by reference in their entirety.

### FIELD OF THE INVENTION

The present disclosure relates generally to medical-device data communication, LoRa and LoRa mesh communication, patient-associated device connectivity, clinical data validation, and downstream clinical system integration.

More particularly, the disclosure relates to systems, devices, methods, and computer-implemented workflows for transmitting medical-device data, physiological data, alarm data, device-status data, device-identity data, responder-status data, or other patient-associated or clinically relevant data through LoRa or LoRa mesh communication using encrypted packets that include relay-readable routing information, protected clinical-context metadata, and encrypted payload data.

The disclosure further relates to authorized gateway systems configured to validate LoRa-originated packet information against authorization records and patient, device, location, gateway, session, incident, or downstream-destination context records before releasing decrypted data to an electronic medical record system, medical device data system, medical data governance system, incident command system, or other authorized downstream system.

### BACKGROUND OF THE INVENTION

Medical devices and patient-associated devices increasingly generate data outside the fixed bedside environment. Monitors, pumps, ventilators, wearable sensors, oxygen devices, smart beds, smart IV poles, patient phones, transport monitors, and other devices may produce physiological data, alarm data, device-status data, powered-state data, battery data, device-use data, device-identity data, and other clinically relevant information. This data may be needed during bedside monitoring, patient movement, transport, home care, remote care, emergency response, disaster response, and other settings where conventional communication paths are unreliable or unavailable.

Existing clinical communication systems often rely on wired networking, Wi-Fi, Bluetooth, cellular networks, proprietary telemetry, or local bedside gateways. These systems may be effective in fixed locations, but they can become unreliable during patient transport, building movement, network outage, power interruption, remote monitoring, emergency operations, or operation in structures where radio coverage is degraded. Short-range systems such as Bluetooth or Bluetooth Low Energy may lose connectivity when a patient or device leaves the range of a phone, bedside gateway, or local proxy node. Wi-Fi and cellular systems may be unavailable, congested, blocked, or inappropriate in some clinical or emergency environments.

LoRa and LoRa mesh communication can provide low-power, long-range, and off-cellular-network transmission. General-purpose LoRa mesh systems may relay encrypted packets through intermediate nodes and may operate without continuous Wi-Fi or cellular coverage. Such systems, however, are generally designed for generic messaging, tracking, telemetry, or off-grid communication. They do not, by themselves, ensure that medical-device data is associated with the correct patient, device, care location, gateway, session, or downstream clinical destination.

Medical-device data is not clinically useful merely because it reaches a receiver. Before data is released to an electronic medical record system, medical device data system, medical data governance system, clinical command system, or other downstream clinical system, the data should be associated with the correct source device, patient context, device context, location context, gateway context, session context, and downstream authorization. A packet that is technically deliverable over a radio path may still be clinically unsafe if it is associated with the wrong patient, an unexpected device, an expired session, an unauthorized gateway, or an incorrect downstream destination.

General-purpose mesh relay nodes also create a separation problem. Intermediate relay nodes may be useful for extending communication range, but they should not become access points for patient-identifying information, clinical context, device identity, or medical payload data. A communication architecture for medical use should allow relay nodes to forward packets while limiting their access to protected clinical-context metadata and encrypted medical payload data.

Existing medical-device gateways may convert bedside device data into downstream formats such as HL7, FHIR, proprietary medical-device messages, or electronic medical record messages. Such gateways, however, are typically designed for direct or local device connectivity. They do not necessarily provide a packet architecture in which LoRa relay nodes can forward medical-device packets without readable access to clinical context or payload data, while an authorized gateway validates source authorization and patient/device context before downstream clinical release.

Accordingly, there remains a need for a medical-device LoRa communication system that separates transport routing from clinical-context access, allows LoRa relay nodes to forward encrypted packets without reading protected clinical-context metadata or encrypted payload data, and uses an authorized gateway to validate authorization and clinical context before releasing decrypted data to downstream clinical systems.

### SUMMARY OF THE INVENTION

The present disclosure provides a medical-device LoRa communication system configured to transmit medical-device data, physiological data, alarm data, device-status data, device-identity data, or other patient-associated data through a LoRa communication path while preserving clinical context and controlling downstream clinical release.

In one embodiment, the system includes a LoRa communication module coupled to a host medical device or patient-associated medical device. The LoRa communication module obtains source data from the host medical device or patient-associated medical device and generates an encrypted LoRa packet for transmission to an authorized gateway, either directly or through one or more LoRa relay nodes.

The encrypted LoRa packet may include a relay header, protected clinical-context metadata, and an encrypted payload. The relay header is readable by LoRa relay nodes and includes routing information used to forward the packet toward the authorized gateway. The protected clinical-context metadata includes patient-context, device-context, location-context, gateway-context, session-context, or downstream-destination information and is inaccessible in readable form to the LoRa relay nodes. The encrypted payload includes medical-device data, physiological data, alarm data, device-status data, device-identity data, or other patient-associated data.

The LoRa relay nodes are configured to forward the encrypted LoRa packet using the relay header without decrypting the encrypted payload and without accessing the protected clinical-context metadata in readable form. The relay nodes may operate as fixed nodes, mobile nodes, anchor nodes, mesh nodes, repeaters, vehicle-mounted nodes, store-and-forward nodes, or deployable nodes. The relay nodes may update relay-readable routing information, store packets, retransmit packets, suppress duplicates, or assist route recovery without becoming clinical-data access points.

The authorized gateway receives the encrypted LoRa packet and validates packet information before downstream clinical release. The gateway may validate source authorization, destination authorization, encryption-key status, packet sequence, patient context, device context, location context, gateway context, session context, or downstream destination using one or more authorization records and context records.

When validation is unsuccessful, the authorized gateway prevents downstream clinical routing of the packet. The packet may be rejected, quarantined, buffered, separately logged, flagged, or retained for later correction, reconciliation, or audit. When validation is successful, the authorized gateway decrypts the encrypted payload, binds the decrypted data to a validated context record, converts the data into a downstream clinical message, and makes the data available to an authorized downstream system.

The authorized downstream system may include an electronic medical record system, medical device data system, medical data governance system, browser interface, hospital operations system, transport dashboard, clinical command system, cloud system, incident command system, responder-status dashboard, or other authorized clinical, operational, command, or governance system. The downstream message may be formatted as an HL7 message, FHIR resource, JSON object, XML object, MQTT message, HTTPS message, proprietary medical-device message, EMR message, MDDS message, medical data governance message, or another downstream-compatible message.

In some embodiments, the system supports store-and-forward operation, delayed-packet reconciliation, duplicate-packet suppression, stale-packet detection, replay-suspect detection, out-of-order packet handling, missing-sequence detection, and backfill of locally stored source-device data. Delayed or backfilled data may be time-synchronized using original event timestamps and packet sequence values before downstream clinical release.

In some embodiments, the system supports transition between short-range communication and LoRa communication. For example, a patient-associated medical device may communicate through Bluetooth, Bluetooth Low Energy, a bedside gateway, a local proxy network, or another short-range path when local coverage is available, and may transition to LoRa communication when the short-range path is unavailable, degraded, or out of range. The same patient, device, location, gateway, and session context may be maintained across the communication transition.

In some embodiments, the same architecture is applied to emergency responder monitoring. A responder-carried LoRa module obtains responder-status data from responder equipment, oxygen apparatus, self-contained breathing apparatus, motion sensor, fall sensor, temperature sensor, biometric sensor, environmental sensor, manual distress input, or other responder-status source. The responder-carried module generates an encrypted responder-status LoRa packet that is forwarded through fixed, deployable, vehicle-mounted, or perimeter LoRa nodes to an incident command gateway. The incident command gateway validates responder and incident context before displaying or routing responder-status information.

The disclosed architecture separates packet transport from clinical release. LoRa relay nodes provide communication range, redundancy, mesh forwarding, or store-and-forward capability. The authorized gateway controls whether LoRa-originated data is validly associated with the correct source, patient, device, location, session, incident, gateway, or downstream destination before the data is released to clinical, operational, governance, or command systems.

### BRIEF DESCRIPTION OF THE DRAWINGS

FIG. 1 illustrates an overall medical-device LoRa communication architecture including a LoRa communication module, one or more LoRa relay nodes, an authorized gateway, and one or more authorized downstream systems.

FIG. 2 illustrates an example LoRa communication module configured for coupling to a host medical device or patient-associated medical device.

FIG. 3 illustrates an example local interface and patient/device/context association workflow for associating source data with patient, device, location, gateway, or session context.

FIG. 4 illustrates an example encrypted LoRa packet structure including a relay header, protected clinical-context metadata, and an encrypted payload.

FIG. 5 illustrates an example relay-node forwarding workflow in which one or more LoRa relay nodes forward an encrypted LoRa packet without decrypting the encrypted payload or accessing protected clinical-context metadata in readable form.

FIG. 6 illustrates an example authorized-gateway validation workflow including packet validation, routing prevention, quarantine or logging, decryption, context binding, downstream conversion, routing, and audit generation.

FIG. 7 illustrates an example store-and-forward and delayed-packet reconciliation workflow for handling unavailable gateways, unavailable downstream systems, duplicate packets, delayed packets, missing sequences, and backfilled source-device data.

FIG. 8 illustrates an example emergency responder LoRa monitoring architecture including a responder-carried LoRa module, responder-status interfaces, LoRa anchor or relay nodes, an incident command gateway, and an incident command display.

### DETAILED DESCRIPTION

### Definitions and Core Concepts

For purposes of this disclosure, “LoRa” refers to a low-power, long-range wireless communication technique, modulation scheme, radio interface, protocol, or compatible communication system suitable for transmitting data over a range greater than typical short-range communication systems. The term may include LoRa, LoRaWAN, proprietary LoRa-compatible communication, private LoRa communication, point-to-point LoRa communication, LoRa mesh communication, or other long-range low-power radio communication.

The term “LoRa mesh” refers to a communication arrangement in which LoRa-capable modules, relay nodes, anchor nodes, repeaters, gateways, or other nodes may receive, forward, relay, store, retransmit, or route LoRa-originated packets toward an authorized gateway or destination.

A “LoRa communication path” is a direct, relayed, mesh, private, gateway-directed, anchor-assisted, vehicle-assisted, store-and-forward, or point-to-point LoRa path used to move a packet from a LoRa communication module toward an authorized gateway.

A “LoRa communication module” means a LoRa-capable communication component configured to obtain data from, or be associated with, a host medical device or patient-associated medical device and to generate or transmit an encrypted LoRa packet. A LoRa communication module may be implemented as a dongle, embedded board, patch, label, chip, gateway card, wearable bridge, plug-in module, adapter, cartridge, replaceable insert, or integrated radio component.

The phrase “host medical device” refers to a medical, clinical, monitoring, treatment-support, transport, or patient-care device that produces, stores, displays, measures, or communicates medical-device data, physiological data, alarm data, device-status data, device-identity data, or related information.

A “patient-associated medical device” refers to a device associated with a patient, patient encounter, patient location, patient sensor, patient wearable, patient phone, patient monitor, patient bed, patient transport device, or other patient-related source of data. A patient-associated medical device may be temporarily or permanently associated with a patient during a session, procedure, transport event, monitoring interval, admission, discharge, transfer, home-care event, or other care event.

The term “source data” includes data obtained from a host medical device, patient-associated medical device, sensor, wearable, phone, monitor, pump, ventilator, oxygen apparatus, smart bed, smart IV pole, transport monitor, responder device, or other source. Source data may include medical-device data, physiological data, alarm data, powered-state data, battery data, device-use data, device-identity data, device-context data, waveform data, vital-sign data, responder-status data, or patient-input data.

An “encrypted LoRa packet” is a LoRa packet generated for transmission through a LoRa communication path and including at least an encrypted payload. In some embodiments, an encrypted LoRa packet includes a relay header, protected clinical-context metadata, and an encrypted payload.

A “relay header” refers to a relay-readable portion of an encrypted LoRa packet containing information used by relay nodes to forward, store, retransmit, prioritize, suppress, or route the packet. A relay header may include routing information, source-module information, destination-gateway information, packet sequence information, hop count, time-to-live value, packet priority, packet type, checksum, or related transport information.

“Protected clinical-context metadata” refers to metadata associated with a patient, device, location, gateway, session, downstream destination, authorization state, incident, responder, or clinical context that is protected from readable access by one or more LoRa relay nodes. The protected clinical-context metadata may be encrypted, signed, tokenized, pseudonymized, gateway-resolvable, gateway-readable only, or otherwise unavailable in readable form to intermediate relay nodes.

An “encrypted payload” is the encrypted portion of a packet containing source data, medical-device data, physiological data, alarm data, device-status data, device-identity data, responder-status data, or other protected data. The encrypted payload is not readable by LoRa relay nodes unless such relay nodes are also configured as authorized gateways or otherwise authorized endpoints.

A “relay-readable packet portion” means the portion of a packet that a relay node may read or use for transport operations. The relay-readable packet portion may include the relay header and any other information required to forward, prioritize, suppress, or store the packet.

A “relay-unreadable packet portion” means the portion of a packet that is not accessible in readable form to a relay node. The relay-unreadable packet portion may include protected clinical-context metadata, encrypted payload data, patient-identifying information, device-context information, responder-context information, or clinical data.

The term “LoRa relay node” refers to a LoRa-capable node configured to receive and forward an encrypted LoRa packet toward an authorized gateway. A LoRa relay node may be fixed, mobile, vehicle-mounted, room-mounted, bed-area-mounted, wearable, deployable, gateway-adjacent, temporary, or otherwise positioned, and may operate as a relay, anchor, repeater, mesh node, store-and-forward node, perimeter node, transport-route node, or other forwarding element.

An “authorized gateway” is a gateway, receiver, server, router, room gateway, bed gateway, hospital gateway, vehicle gateway, home gateway, cloud gateway, incident command gateway, medical device data system gateway, medical data governance gateway, or other gateway configured to receive, validate, decrypt, bind, convert, route, display, store, quarantine, audit, or govern LoRa-originated data.

The term “authorization record” means a stored or generated record, file, table, token, policy, key association, credential, rule, or data structure used to determine whether a source, device, module, gateway, communication path, encryption key, packet, user role, or downstream destination is authorized.

The term “context record” means a stored or generated record, file, table, token, object, reference, metadata set, rule set, or data structure that preserves an association between LoRa-originated data and one or more patients, devices, modules, rooms, beds, care locations, sessions, gateways, incidents, procedures, transport events, downstream destinations, or governance policies.

A “patient-context record” is a context record associated with a patient, patient identifier, encounter, admission, session, care event, room, bed, assigned device, assigned module, gateway, or downstream destination.

A “device-context record” is a context record associated with a host medical device, patient-associated medical device, LoRa communication module, device identifier, serial number, regulated-device identifier, manufacturer identifier, model identifier, firmware version, powered state, alarm state, maintenance state, calibration state, assigned patient, assigned room, assigned bed, or assigned gateway.

A “care-location record” is a context record associated with a room, bed, floor, department, care area, transport route, ambulance bay, home-care location, operating room, imaging suite, emergency department, field location, or other physical or logical care location.

A “session record” is a context record defining a time-bounded or event-bounded association, such as a monitoring session, procedure, transport event, patient room stay, home-care interval, alarm episode, device-use interval, emergency incident, responder assignment, or other clinical or operational period.

The terms “clinical release” and “downstream clinical release” refer to making decrypted, validated, or context-bound data available to a downstream clinical, operational, command, governance, display, or storage system as usable data. Clinical release may include routing, transmitting, publishing, displaying, storing, exporting, converting, forwarding, presenting, or otherwise providing data after validation.

A “downstream system” is a system that receives, stores, displays, analyzes, routes, governs, or acts upon data released by an authorized gateway. A downstream system may include an electronic medical record system, medical device data system, medical data governance system, browser interface, clinical command system, hospital operations system, transport dashboard, biomed system, maintenance system, cloud system, incident command system, responder-status dashboard, or other authorized destination.

A “downstream message” is a message generated from validated and context-bound data for delivery to a downstream system. A downstream message may be formatted as an HL7 message, FHIR resource, JSON object, XML object, MQTT message, HTTPS message, TCP/IP message, UDP message, proprietary medical-device message, EMR message, MDDS message, medical data governance message, browser-renderable message, hospital operations message, or incident-command message.

“Gateway validation” refers to one or more operations performed by an authorized gateway to determine whether a packet may be decrypted, bound, converted, routed, displayed, stored, audited, or released downstream. Gateway validation may include source authorization, destination authorization, encryption-key validation, packet-signature validation, packet-sequence validation, patient-context validation, device-context validation, location-context validation, session validation, incident validation, responder-context validation, gateway validation, or downstream-destination validation.

“Clinical release control” refers to preventing or permitting downstream clinical release based on gateway validation, context validation, authorization records, governance rules, packet state, user confirmation, or another release condition.

The term “quarantine” refers to storing, retaining, isolating, marking, buffering, suppressing, or separately logging a packet or data item that failed validation, requires confirmation, appears mismatched, is delayed, is stale, is replay-suspect, is duplicate, or is otherwise not ready for downstream clinical release.

An “audit record” is a record of one or more packet, validation, routing, display, correction, quarantine, decryption, binding, conversion, release, reconciliation, or governance events. An audit record may include timestamps, source identifiers, device identifiers, gateway identifiers, context references, packet sequence values, validation results, downstream destinations, user identifiers, and actions taken.

A “packet lifecycle audit record” is an audit record that tracks a packet from receipt or generation through validation, validation failure, quarantine, decryption, context binding, conversion, routing, downstream release, suppression, reconciliation, or final disposition.

A “source-module identifier” means an identifier, token, credential, serial number, cryptographic reference, address, or other value identifying or representing the LoRa communication module or responder-carried LoRa module that generated or transmitted a packet.

A “device identifier” means an identifier, token, serial number, regulated-device identifier, manufacturer identifier, model identifier, equipment identifier, or other value identifying or representing a host medical device, patient-associated medical device, sensor, monitor, pump, ventilator, oxygen apparatus, wearable, phone, bed, or other device.

A “destination-gateway identifier” means an identifier, token, address, credential, gateway class, gateway group, facility identifier, room-gateway identifier, vehicle-gateway identifier, incident-command-gateway identifier, or other value identifying or representing an intended gateway or gateway group.

An “encryption-key identifier” means a value, token, field, reference, or credential identifying or representing an encryption key, session key, device key, patient-context key, module key, gateway key, responder key, rotating key, or other cryptographic association.

A “packet sequence value” means a number, counter value, token, timestamp-derived value, unique message value, or other ordering value assigned to a packet and used for duplicate detection, missing-sequence detection, delay detection, replay-suspect detection, out-of-order detection, reconciliation, or audit.

“Store-and-forward” refers to storing a packet or source data when a communication path, relay node, gateway, or downstream system is unavailable and forwarding the packet or data when communication becomes available.

“Data-gap detection” refers to identifying a missing packet, missing sequence value, missing time interval, missing measurement, missing source-data segment, or missing downstream data segment associated with a device, patient, session, context record, gateway, or downstream data stream.

“Backfill” refers to requesting, retrieving, transmitting, validating, time-synchronizing, and inserting locally stored source-device data corresponding to a missing packet, missing time interval, missing sequence value, or other data gap.

“Responder-status data” means information associated with an emergency responder, firefighter, emergency medical technician, police officer, hazmat worker, search-and-rescue worker, military medic, industrial safety worker, or other responder. Responder-status data may include responder identity, team assignment, incident identifier, location, motion state, no-motion state, fall state, vital-sign state, oxygen status, temperature exposure, battery state, distress state, Mayday state, environmental state, or equipment status.

An “incident command gateway” is an authorized gateway used in an emergency response, incident command, disaster response, fireground, industrial safety, rescue, military, field, or command environment to receive, validate, decrypt, bind, display, route, audit, or govern responder-status data.

A “medical data governance system” is one or more systems configured to validate, bind, route, audit, reconcile, control access to, apply policy to, preserve context for, or govern medical, clinical, device, responder, location, session, incident, or operational data.

A “patient-associated data” refers to data that is linked directly or indirectly to a patient, patient identifier, encounter, room, bed, procedure, care event, patient-associated device, wearable, sensor, phone, monitor, or downstream clinical record. Patient-associated data may include medical-device data, physiological data, alarm data, device-status data, device-identity data, responder-status data when clinically associated with patient care, or operational data associated with a patient care event.

A “local communication interface” is a wired, wireless, optical, sensor, user-actuated, or software interface by which a LoRa communication module obtains source data, identifiers, pairing information, provisioning data, or context information from a host medical device, patient-associated medical device, sensor, phone, application, clinician pairing device, or user.

“Relay metadata” means information generated, appended, stored, or forwarded by a LoRa relay node during packet receipt, storage, retransmission, route selection, or forwarding. Relay metadata may include a relay-node identifier, timestamp, signal-quality value, hop-path value, retry counter, store-and-forward state, route-table state, received-power value, or link-quality value.

A “route table” is a data structure stored by a LoRa relay node, LoRa communication module, gateway, or other node that associates a destination, destination gateway, route class, final node, or gateway group with a next-hop node, relay path, relay mode, route state, timestamp, signal-quality value, or routing priority.

“Time synchronization” refers to aligning source data, delayed data, backfilled data, packet data, or downstream messages using original event timestamps, received timestamps, device clocks, gateway clocks, sequence values, session identifiers, care-location records, or medical data governance time references.

Unless otherwise indicated, “medical-device data,” “physiological data,” “alarm data,” “device-status data,” “device-identity data,” and “patient-associated data” may overlap. A device identifier, alarm state, powered-state value, battery value, sensor value, waveform value, vital-sign value, or device-use value may be both device-associated and patient-associated when the device is assigned to, worn by, connected to, used near, or otherwise associated with a patient, encounter, room, bed, care event, session, or procedure.

Unless the context requires otherwise, “protected metadata,” “protected clinical-context metadata,” and similar phrases include metadata that is encrypted, signed, tokenized, pseudonymized, access-controlled, gateway-resolvable, gateway-readable only, or otherwise unavailable in readable form to one or more LoRa relay nodes. Protected metadata need not be protected from every component in the system. In some embodiments, protected metadata is specifically protected from intermediate relay nodes while remaining accessible to an authorized gateway.

For purposes of validation, the phrases “valid patient context,” “valid device context,” “valid location context,” “valid session context,” “valid incident context,” and similar phrases mean that packet information corresponds to one or more stored or generated context records, authorization records, association records, gateway records, downstream-destination records, or governance rules sufficient to permit a defined system action, such as decryption, binding, conversion, routing, display, storage, audit, or downstream clinical release.

Unless expressly stated otherwise, “downstream routing,” “downstream clinical routing,” “release,” “make available,” and similar phrases include transmitting, publishing, displaying, storing, exporting, converting, forwarding, presenting, or otherwise providing validated data or a downstream message to an authorized downstream system, user interface, clinical system, command system, operational system, governance system, or storage system.

When used with respect to packets or data, the terms “delayed,” “stale,” “replay-suspect,” “duplicate,” and “out-of-order” refer to packet-handling classifications used for validation, reconciliation, suppression, warning, quarantine, audit, or downstream-routing decisions. Such classifications do not necessarily require that the packet or data be discarded.

### Architecture of the Medical-Device LoRa Communication System

In one embodiment, the system includes a LoRa communication module coupled to a host medical device or to a patient-associated medical device. The host medical device may be, for example, a monitor, pump, ventilator, oxygen apparatus, smart bed, transport monitor, wearable sensor, medical patch, patient phone, smart IV pole, or other device that produces medical-device data, physiological data, alarm data, device-status data, device-identity data, or related patient-associated data.

The LoRa communication module operates as the local acquisition and packet-generation point. It receives source data from the host medical device or patient-associated medical device through a local communication interface and generates an encrypted LoRa packet. The packet is configured for transmission over a LoRa communication path to an authorized gateway, either directly or through one or more LoRa relay nodes.

The LoRa communication path may be a direct point-to-point LoRa path, a private LoRa mesh path, a relay-node path, an anchor-node path, a store-and-forward path, or a combination of such paths. The system does not require that every intermediate node be trusted to read clinical information. Instead, the relay nodes are used for transport. They forward packets using relay-readable routing information and are prevented from accessing protected clinical-context metadata or encrypted medical payload data in readable form.

The authorized gateway is the control point between LoRa-originated traffic and downstream clinical use. The authorized gateway stores or accesses authorization records and context records. The context records may include patient-context records, device-context records, care-location records, gateway records, session records, and downstream-destination records. Before LoRa-originated data is released to a downstream clinical system, the authorized gateway validates packet information against the applicable authorization and context records.

If the validation fails, the authorized gateway prevents downstream clinical routing. The packet may be rejected, quarantined, buffered, separately logged, flagged, or retained for reconciliation or audit. In this condition, the packet may remain transport evidence or a technical communication record, but it is not released as clinically valid data to an electronic medical record system, medical device data system, medical data governance system, or other downstream clinical destination.

If the validation succeeds, the authorized gateway decrypts the payload, associates the decrypted payload data with the applicable context record, and makes the data available to an authorized downstream system. The downstream system may include an electronic medical record system, medical device data system, medical data governance system, browser interface, hospital operations system, transport dashboard, clinical command system, cloud system, or another authorized clinical or operational system.

In some embodiments, the authorized gateway converts the context-associated payload data into a downstream message. The downstream message may be formatted as an HL7 message, FHIR resource, JSON object, XML object, MQTT message, HTTPS message, proprietary medical-device data message, EMR message, MDDS message, medical data governance message, or another message format required by the receiving system.

The architecture separates communication transport from clinical release. LoRa relay nodes provide range extension, redundancy, mesh routing, or store-and-forward capability. The authorized gateway determines whether the received packet is authorized and clinically associated with the correct patient, device, location, session, or downstream destination. This separation permits use of low-power or long-range LoRa relay infrastructure without treating each relay node as a clinical-data access point.

In one implementation, a medical-device LoRa communication architecture includes: a LoRa communication module at the device side; one or more LoRa relay nodes in the communication path; an authorized gateway at the clinical-network boundary; and one or more authorized downstream systems. The LoRa communication module generates the encrypted packet, the relay nodes transport the packet, and the authorized gateway validates, decrypts, binds, converts, routes, or blocks the packet according to the applicable authorization and context records.

In another implementation, the LoRa communication module and the authorized gateway are provisioned with corresponding identifiers, keys, context references, or authorization settings. The packet may identify a source module, source device, patient context, device context, destination gateway, encryption key, packet sequence, packet priority, or other routing or validation information. The gateway may use this information to distinguish packets that may be released downstream from packets that should be blocked, quarantined, delayed, or reconciled.

The system may operate inside a hospital, during patient transport, in a home-care environment, in a remote-care setting, in a field hospital, during disaster response, or in another environment where Wi-Fi, wired networking, cellular networking, or short-range communication is unavailable, degraded, congested, unsafe, or unreliable. The same architecture may support direct gateway coverage, relay-assisted coverage, mobile relay coverage, vehicle gateway coverage, or temporary coverage established by deployable relay or anchor nodes.

The architecture is not limited to a specific LoRa network server, public LoRaWAN service, cellular backhaul, cloud service, or carrier-operated infrastructure. In some embodiments, the LoRa communication path is private and terminates at an authorized gateway controlled by a healthcare facility, medical data system, emergency command system, medical data governance system, or other authorized entity.

### LoRa Communication Module and Local Medical-Device Interfaces

In one embodiment, the LoRa communication module is a physical or embedded communication component coupled to a host medical device or patient-associated medical device. The module may be implemented as a plug-in dongle, embedded board, patch, label, chip, adapter, gateway card, wearable bridge, cartridge, replaceable insert, or integrated radio module.

The module includes a LoRa transceiver, processor, memory, encryption logic, local communication interface, antenna, and power source. The processor executes firmware or software instructions for acquiring source data, generating packet sequence values, selecting encryption keys, constructing LoRa packets, buffering packets, scheduling transmissions, receiving acknowledgements, and managing retransmission or low-power operation.

The local communication interface provides the connection between the LoRa communication module and the host device. The interface may be a wired medical-device interface, serial interface, USB interface, Bluetooth interface, Bluetooth Low Energy interface, NFC-assisted pairing interface, optical interface, proprietary device interface, sensor interface, or other local communication path. The interface may receive medical-device data, physiological data, alarm data, battery data, powered-state data, device-use data, device-identity data, calibration data, maintenance data, or other source data.

The memory stores or receives local configuration data used to generate and validate LoRa packets. Such data may include a source-module identifier, device identifier, patient-context reference, device-context reference, encryption-key identifier, destination-gateway identifier, packet sequence counter, device profile, routing parameter, packet priority, or downstream destination setting. The stored values may be provisioned during manufacturing, installation, patient admission, device pairing, bedside setup, transport preparation, home-care setup, or gateway assignment.

The encryption logic encrypts payload data before LoRa transmission. The encryption logic may also generate packet signatures, select encryption keys, associate an encryption-key identifier with a packet, protect clinical-context metadata, tokenize identifiers, or support key rotation. The encryption logic may be implemented in software, firmware, hardware, secure memory, cryptographic circuitry, or a combination thereof.

The LoRa transceiver transmits encrypted LoRa packets and may receive acknowledgements, gateway instructions, provisioning data, configuration updates, or relay-state information. The transceiver may operate in direct point-to-point mode, private mesh mode, relay-assisted mode, anchor-assisted mode, store-and-forward mode, or gateway-directed mode. The module may communicate with a single authorized gateway or with multiple authorized gateways depending on the provisioning state and operating environment.

The antenna may be internal or external and may be configured according to the physical form factor of the module and the host device. The antenna may be printed, flexible, embedded, shielded, directional, omnidirectional, detachable, or integrated into the module housing. In some embodiments, antenna configuration is selected based on expected patient transport routes, hospital wall penetration, responder use, field deployment, or home-care range.

The power source may be internal battery power, rechargeable battery power, replaceable battery power, host-device power, USB power, wall power, vehicle power, gateway-supplied power, or another suitable source. The module may switch between host power and internal power when host power is unavailable, interrupted, or insufficient. The module may also report battery state, power fault, estimated remaining life, charging state, or replacement state to the authorized gateway.

The module housing may be sealed, sterilizable, reusable, disposable, waterproof, tamper-resistant, tamper-evident, cleanable, biocompatible, impact-resistant, fire-resistant, or intrinsically safe depending on the intended environment. The housing may include an attachment interface such as a plug, connector, adhesive surface, clip, bracket, snap-fit feature, magnetic coupling, cable interface, housing slot, battery-compartment insert, strap, or rail mount.

In operation, the LoRa communication module receives source data through the local communication interface. The processor assigns a packet sequence value, retrieves or receives the applicable identifiers and context references, selects an encryption key, encrypts the payload, generates relay-readable packet information, generates protected clinical-context metadata, and transmits the resulting encrypted LoRa packet through the LoRa transceiver.

In some embodiments, the module buffers packets when a relay path or authorized gateway is unavailable. Buffered packets may preserve original event time, packet sequence value, source-module identifier, device identifier, and context reference. When communication is restored, the module forwards the stored packets for gateway validation and downstream reconciliation.

The module may also support provisioning or association operations. For example, a clinician may pair the module with a patient, device, room, bed, gateway, or care session using a barcode, QR code, NFC tag, Bluetooth exchange, device cable, hospital application, or clinician pairing device. The resulting context reference may be stored locally in the module, stored at the authorized gateway, stored in a medical data governance system, or distributed among those components.

The LoRa communication module therefore provides the device-side boundary of the system. It converts locally acquired medical-device or patient-associated data into an encrypted LoRa packet that can be transported by LoRa relay infrastructure without exposing protected clinical context or medical payload data to intermediate relay nodes.

### Patient, Device, Location, Gateway, and Session Association

The system uses context association to determine whether LoRa-originated data may be treated as clinically usable data. A packet may be received over a valid radio path but still be blocked from downstream clinical release if the patient, device, location, gateway, session, or downstream destination cannot be validated.

In one embodiment, a patient-context record links a patient identifier to one or more devices, modules, rooms, beds, care locations, sessions, gateways, encryption keys, or downstream destinations. The patient identifier may be obtained from a patient bracelet, barcode, QR code, NFC tag, RFID tag, admission system, electronic medical record system, medical data governance system, patient phone, hospital application, biometric input, or clinician input.

A device-context record links a device identifier to a host medical device, patient-associated medical device, LoRa communication module, device type, serial number, regulated-device identifier, manufacturer identifier, model identifier, firmware version, powered state, alarm state, maintenance state, calibration state, assigned patient, assigned room, assigned bed, or assigned gateway. The device identifier may be read from the device, from the LoRa module, from a device label, from a wired interface, from a wireless interface, or from a hospital equipment record.

A care-location record links a patient, device, module, packet, or session to a room, bed, floor, department, transport route, operating room, imaging suite, emergency department, ambulance bay, home-care location, field location, or other care area. The location may be obtained from a room marker, bed marker, gateway proximity, clinician confirmation, admission/discharge/transfer system, location system, LoRa anchor, Bluetooth beacon, NFC tag, QR code, RFID tag, or another location source.

A gateway record identifies an authorized gateway permitted to receive, validate, decrypt, route, or audit packets for a patient, device, care location, or session. The gateway may be a room gateway, bed gateway, floor gateway, hospital gateway, vehicle gateway, home gateway, medical device data system gateway, medical data governance gateway, incident command gateway, or other authorized receiver.

A session record defines a time-bounded or event-bounded association. A session may correspond to a monitoring interval, transport event, procedure, room stay, home-care period, alarm episode, device-use interval, emergency incident, or other clinical or operational period. The session record may include a start time, stop time, expected device, expected gateway, expected care location, authorized downstream destination, or context status.

The association may be established before data collection, during data collection, or after data collection. In some embodiments, the LoRa communication module is provisioned with a patient-context reference or device-context reference before transmission. In other embodiments, the module transmits a token or module identifier, and the authorized gateway resolves the token against stored context records. In further embodiments, data is collected first and later associated with a patient or device after confirmation by a clinician or another authorized system.

The association event may be created by scanning a patient bracelet, scanning a device code, tapping an NFC tag, detecting a Bluetooth or BLE device, connecting a device cable, reading a serial number, receiving an admission/discharge/transfer message, detecting gateway proximity, or receiving clinician confirmation. The association event may create or update a patient-device association, device-location association, gateway association, or session association.

The system may require one association source or multiple association sources. For example, a patient-device association may be based on a patient bracelet and device identifier. A device-location association may be based on a room identifier and gateway proximity. A session association may be based on a clinician confirmation and a time interval. These sources may be used together to reduce the risk that medical-device data is attributed to the wrong patient or device.

If the association is valid, the system may generate a context reference for inclusion in protected clinical-context metadata or for lookup by the authorized gateway. The context reference may identify a patient-context record, device-context record, care-location record, session record, gateway record, or downstream-destination record. The context reference may be encrypted, signed, tokenized, or otherwise protected so that relay nodes cannot read patient or device context in transit.

If the association is missing, expired, inconsistent, duplicated, or unauthorized, the system may identify an association mismatch condition. Examples include a patient-device mismatch, room mismatch, bed mismatch, gateway mismatch, expired session, unauthorized module, unexpected device, wrong downstream destination, or missing clinician confirmation. In response, the system may prevent downstream clinical release, quarantine the packet, request re-pairing, request confirmation, update an audit record, or store the data for later reconciliation.

In some embodiments, the authorized gateway validates the association before decrypting the payload. In other embodiments, the gateway may access limited protected clinical-context metadata or a gateway-readable context token before payload decryption. In either case, downstream clinical routing is prevented unless the packet can be associated with an authorized source and a valid patient, device, location, or session context.

The context association process is independent of the radio path used to deliver the packet. A packet forwarded by a valid LoRa relay path may still be blocked if the clinical context is invalid. Conversely, a packet delayed or forwarded through multiple relay nodes may be accepted if the packet context, sequence, source, destination, and authorization records validate at the authorized gateway.

This association layer allows the communication system to separate transport success from clinical validity. The relay network determines whether a packet can physically reach a gateway. The authorized gateway determines whether the packet may become clinically usable data.

### Encrypted LoRa Packet Structure

In one embodiment, the LoRa communication module generates an encrypted LoRa packet having three functional portions: a relay header, protected clinical-context metadata, and an encrypted payload. The portions may be contained in a single packet, divided across packet fragments, or represented by fields that are logically separated by encryption, tokenization, signing, access control, or gateway-only interpretation.

The relay header is the portion of the packet used by LoRa relay nodes to move the packet through the communication path. The relay header may include a source-module identifier, destination-gateway identifier, packet sequence value, hop count, time-to-live value, packet priority, packet type, checksum, route class, relay class, packet length, or other routing information. The relay header is readable by relay nodes so that the packet can be forwarded, repeated, stored, retransmitted, prioritized, or discarded without access to clinical content.

The protected clinical-context metadata carries information used by the authorized gateway to associate the packet with a patient, device, location, gateway, session, or downstream destination. The protected clinical-context metadata may include a patient-context reference, device-context reference, care-location reference, room identifier, bed identifier, session identifier, gateway identifier, encryption-key identifier, downstream-destination identifier, access-control rule identifier, routing-rule identifier, quarantine-rule identifier, audit-rule identifier, event timestamp, or other context information.

The protected clinical-context metadata is not readable by LoRa relay nodes. It may be encrypted, signed, tokenized, pseudonymized, wrapped for gateway access, or otherwise protected. In some embodiments, a relay node may detect that protected clinical-context metadata is present but cannot determine the patient, device, room, bed, session, or downstream clinical destination represented by the metadata.

The encrypted payload contains the medical-device or patient-associated data. The payload may include medical-device data, physiological data, alarm data, powered-state data, battery data, device-use data, device-identity data, waveform data, vital-sign data, infusion data, ventilation data, pulse oximetry data, ECG data, blood pressure data, glucose data, temperature data, capnography data, oxygen data, patient-input data, or other data obtained from the host medical device or patient-associated medical device.

The packet may include a packet signature, checksum, message authentication code, certificate reference, or other integrity element. A checksum may be relay-readable so that corrupted packets can be discarded before further transmission. A packet signature or message authentication code may be validated by the authorized gateway to detect alteration, unauthorized source, invalid key state, replay, or other packet-integrity failure.

The packet sequence value may be generated by a packet sequence counter at the LoRa communication module. The sequence value may be used by relay nodes for duplicate suppression and by the authorized gateway for duplicate detection, missing-sequence detection, delayed-packet detection, replay-suspect detection, out-of-order detection, reconciliation, and audit. The sequence value may be unique per source module, per device, per session, per patient-context record, or per key interval.

The hop count or time-to-live value limits packet propagation through the LoRa communication path. A relay node may decrement the hop count, update the time-to-live value, append relay metadata, or suppress forwarding when the relay limit has been reached. This prevents indefinite relay propagation and allows different packet classes to use different forwarding limits.

The packet priority may indicate routine, urgent, alarm, emergency, delayed, retransmitted, store-and-forward, configuration, association, or acknowledgement traffic. The priority may affect relay scheduling, retransmission interval, route selection, store-and-forward behavior, gateway processing order, or downstream release handling.

In some embodiments, the packet includes a relay-readable portion and a relay-unreadable portion. The relay-readable portion includes the relay header and any fields needed for transport. The relay-unreadable portion includes the protected clinical-context metadata and encrypted payload. This allows intermediate relay nodes to participate in delivery without becoming access points for clinical context or patient data.

In some embodiments, the protected clinical-context metadata and encrypted payload are encrypted using the same key. In other embodiments, the metadata and payload use different keys, different wrappers, different signing rules, or different access policies. For example, protected clinical-context metadata may be readable only by a class of authorized gateways, while the encrypted payload may be decryptable only by a specific authorized gateway after context validation.

In some embodiments, the destination-gateway identifier identifies a particular gateway. In other embodiments, it identifies a gateway class, facility, department, room group, vehicle gateway, home gateway, command gateway, medical device data system gateway, or medical data governance gateway. The gateway may accept the packet only if the destination-gateway information corresponds to an authorization record or a valid context record.

The encrypted LoRa packet may be transmitted directly to an authorized gateway or through one or more relay nodes. The packet structure remains compatible with direct transmission, mesh transmission, broadcast relay, unicast relay, route recovery, store-and-forward delivery, and delayed reconciliation. The relay nodes use the relay header. The authorized gateway uses the protected clinical-context metadata and payload after validation.

This packet structure separates transport handling from clinical interpretation. Relay nodes receive enough information to forward the packet, but not enough information to determine patient identity, device context, clinical content, or downstream clinical use. The authorized gateway performs the clinical-context validation and controls whether the payload is decrypted and released downstream.

### Relay-Node Forwarding, Mesh Routing, and Route Recovery

In one embodiment, a LoRa relay node receives an encrypted LoRa packet and forwards the packet toward an authorized gateway using relay-readable information in the relay header. The relay node does not decrypt the encrypted payload and does not access protected clinical-context metadata in readable form.

A relay node may be fixed, mobile, vehicle-mounted, room-mounted, bed-area-mounted, wearable, deployable, gateway-adjacent, or temporarily installed. A relay node may operate as a relay, anchor, repeater, mesh node, store-and-forward node, perimeter node, transport-route node, or other LoRa-capable forwarding element.

When a relay node receives an encrypted LoRa packet, it reads the relay header to determine whether the packet should be forwarded. The relay node may evaluate a destination-gateway identifier, packet sequence value, hop count, time-to-live value, packet priority, packet type, checksum, relay class, or route class. If forwarding is permitted, the relay node may update the hop count or time-to-live value, append relay metadata, and retransmit the packet.

Relay metadata may include a relay-node identifier, timestamp, signal-quality value, hop-path value, received signal strength, link-quality indicator, battery state, store-and-forward state, retry counter, or other relay-observable information. Relay metadata may be included in a relay-readable extension, stored locally, or forwarded to the authorized gateway. Relay metadata does not require the relay node to decrypt the payload or read protected clinical-context metadata.

A relay node may forward a packet using unicast transmission, broadcast transmission, or both. If the relay node has a route-table entry for the destination gateway or next hop, the relay node may transmit the packet by unicast. If no route-table entry is available, if a route-table entry has expired, or if a unicast acknowledgement is not detected, the relay node may transmit the packet by broadcast.

A route table may associate a destination, destination gateway, gateway group, route class, or final node with a next-hop node. The route table may be stored locally at the relay node and may be updated based on acknowledgements, observed retransmissions, signal quality, route success, route failure, or gateway response.

An acknowledgement frame may indicate that a packet was received, forwarded, accepted, or delivered. The acknowledgement frame may include relay-readable packet-identifying information, such as a source-module identifier, destination-gateway identifier, packet sequence value, packet type, or checksum. The acknowledgement frame may allow a relay node to update a route-table entry without learning protected clinical-context metadata or encrypted payload data.

A relay node may also update routing information by observing retransmissions from neighboring relay nodes. If a neighboring node retransmits a packet having a matching packet sequence value or other relay-readable identifier, the observing relay node may determine that the neighboring node is a usable next hop for that destination or route class. The relay node may then add, refresh, rank, or remove a route-table entry.

If a unicast attempt fails, the relay node may remove or mark stale the next-hop entry and fall back to broadcast transmission. If a later acknowledgement or observed retransmission indicates that the route is restored, the relay node may update the route table and resume unicast transmission for later packets. This provides route recovery when a node moves, loses power, leaves range, becomes obstructed, or otherwise becomes unavailable.

To reduce redundant transmissions, a relay node may suppress duplicate packets. Duplicate detection may be based on packet sequence value, source-module identifier, destination-gateway identifier, packet signature, checksum, timestamp, or another relay-readable value. If the relay node receives or observes a packet it has already forwarded or scheduled for forwarding, the relay node may cancel its retransmission or store only a duplicate-reception record.

A relay node may use a random or scheduled delay before broadcast retransmission. During the delay, the relay node may listen for the same packet being retransmitted by another relay node. If an equivalent packet is detected, the relay node may suppress its own broadcast. This reduces collision risk, network congestion, and unnecessary power use.

In some embodiments, a packet priority affects relay behavior. A routine packet may use a limited retry count, lower priority queue, lower hop limit, or longer retransmission interval. An urgent or alarm packet may use a higher priority queue, shorter retry interval, increased hop limit, multiple relay paths, or broadcast fallback. Packet priority may be assigned by the LoRa communication module and may be adjusted by relay rules without exposing protected clinical content.

A relay node may temporarily store packets when a next-hop node or authorized gateway is unavailable. Stored packets may retain the original relay header, protected clinical-context metadata, encrypted payload, packet sequence value, timestamp, and relay metadata. When a route becomes available, the relay node forwards the stored packet. The relay node may mark the packet or relay event as delayed or store-and-forwarded.

In some embodiments, relay nodes form a private LoRa communication path that does not require a cellular network, public Internet connection, carrier-operated infrastructure, or public LoRaWAN server. In other embodiments, a LoRaWAN server, cloud system, private network server, vehicle gateway, hospital gateway, or home gateway may be used, provided that clinical-context validation and downstream release control remain under control of an authorized gateway or authorized downstream system.

The relay-node layer therefore performs transport functions, not clinical release functions. It may receive, forward, repeat, store, prioritize, suppress, and recover packets. The authorized gateway remains responsible for validating source, destination, context, and downstream release.

### Authorized Gateway Validation and Clinical Release Control

In one embodiment, the authorized gateway is the boundary between LoRa packet transport and downstream clinical use. A packet may reach the gateway through a valid LoRa path, but the packet is not released downstream as clinical data until the gateway validates the packet against applicable authorization and context records.

The authorized gateway receives an encrypted LoRa packet from a LoRa communication module directly or through one or more LoRa relay nodes. The packet may include a relay header, protected clinical-context metadata, and an encrypted payload. The gateway may read packet information needed for validation, including source information, destination information, packet sequence information, packet signature, encryption-key information, protected context references, or gateway-readable metadata.

The gateway stores or accesses authorization records and context records. Authorization records may identify authorized source modules, authorized devices, authorized gateways, valid encryption keys, valid packet signatures, permitted communication paths, permitted downstream systems, or permitted user roles. Context records may identify patient, device, location, bed, room, gateway, session, encounter, procedure, transport event, or downstream destination relationships.

The gateway may perform validation before decrypting the payload. In some embodiments, the gateway validates source authorization, destination authorization, encryption-key authorization, packet sequence, patient context, device context, location context, gateway context, session context, and downstream destination. In other embodiments, the gateway first accesses a limited gateway-readable portion of protected clinical-context metadata and then performs the validation before releasing any decrypted clinical payload downstream.

Source authorization may confirm that the source module, source device, or associated key is recognized and permitted to transmit into the system. Destination authorization may confirm that the packet is intended for the receiving gateway, gateway group, facility, department, room, vehicle, home gateway, medical device data system gateway, or medical data governance gateway.

Encryption-key validation may confirm that the key identifier, key state, certificate, token, packet signature, or cryptographic association is valid. A packet may be rejected or blocked when a key is expired, revoked, mismatched, absent, or not associated with the claimed source or context.

Packet-sequence validation may confirm that the packet sequence value is expected for the source module, device, session, or context record. The gateway may detect duplicate packets, missing packets, delayed packets, stale packets, replay-suspect packets, or out-of-order packets. Sequence validation may be used for routing decisions, reconciliation, warning flags, duplicate suppression, or audit.

Patient-context validation may confirm that the packet corresponds to a valid patient-context record. Device-context validation may confirm that the packet corresponds to a valid device-context record. Location validation may confirm that the packet is consistent with the expected room, bed, care location, gateway, or transport route. Session validation may confirm that the packet falls within an active monitoring session, procedure, transport event, home-care interval, or other authorized time period.

If validation is unsuccessful, the gateway prevents downstream clinical release. The packet may be rejected, quarantined, buffered, separately logged, flagged, routed for confirmation, or retained for later reconciliation. The gateway may also generate an audit record identifying the packet, source, destination, sequence value, failed validation condition, gateway, timestamp, and action taken. In this state, the packet may remain a technical communication record, but it is not made available as clinically valid data to the downstream clinical system.

If validation is successful, the gateway decrypts the encrypted payload. The gateway binds the decrypted payload data to the validated context record. Binding may associate the data with a patient, device, room, bed, care location, gateway, session, encounter, procedure, transport event, or downstream destination. The gateway may then convert, route, display, store, or otherwise make the data available to an authorized downstream system.

The gateway may maintain a clinical release authorization state for each packet or packet group. The release state may indicate blocked, quarantined, pending confirmation, delayed, duplicate-suppressed, warning-marked, validated, decrypted, routed, or released. The release state may be stored in an audit record, packet lifecycle record, medical data governance system, gateway log, or downstream system.

In some embodiments, the gateway does not route the original packet downstream. Instead, it creates a downstream message based on the validated and context-bound payload data. The downstream message may include original event time, received time, patient-context reference, device-context reference, device identifier, alarm state, source module, gateway identifier, validation result, and one or more payload values. The downstream message may also carry flags indicating delayed delivery, duplicate suppression, replay suspicion, backfilled data, or manual confirmation.

The authorized gateway may also enforce governance rules. Governance rules may specify which downstream systems may receive data, which users may view or correct data, which packets must be quarantined, which alarms require immediate routing, which delayed packets require warning labels, which associations require confirmation, and which packets may be exported or retained. Governance rules may be applied before decryption, after decryption, before downstream conversion, or before clinical release.

The gateway may also support limited bidirectional functions. For example, the gateway may send provisioning data, key updates, route settings, pairing instructions, acknowledgement messages, retry settings, packet-priority settings, or context-update messages to a LoRa communication module. In embodiments used with medical devices, such bidirectional functions may be limited to communication, provisioning, routing, alerting, association, security, firmware, audit, or data-governance operations and need not alter a therapeutic function of the host medical device.

The authorized gateway therefore functions as a validation and release-control point. The relay network may deliver a packet, but the gateway determines whether the packet is authorized, clinically associated, decryptable, context-bound, and permitted for downstream clinical release.

### Downstream Conversion, Routing, and Audit Trail

After validation and decryption, the authorized gateway may convert the decrypted and context-bound payload data into a downstream message suitable for an authorized receiving system. The conversion may be performed by the authorized gateway, a medical device data system gateway, a medical data governance system, a hospital interface engine, a cloud gateway, an edge server, or a distributed combination of these components.

The downstream message may be formatted for the receiving system. Suitable formats include HL7, FHIR, JSON, XML, MQTT, HTTPS, TCP/IP, UDP, proprietary medical-device data messages, electronic medical record messages, medical device data system messages, browser-renderable messages, medical data governance messages, hospital operations messages, or other clinical or operational message formats.

The conversion may include field mapping, unit normalization, timestamp assignment, context enrichment, device-identifier insertion, patient-context linking, alarm-state mapping, value filtering, data reduction, waveform packaging, trend generation, or message segmentation. The conversion may preserve both the original event time and the gateway received time. This allows downstream systems to distinguish real-time data from delayed or backfilled data.

In one embodiment, the authorized gateway converts a validated payload from a medical device into a downstream message that includes a device identifier, source-module identifier, patient-context reference, device-context reference, original event timestamp, received timestamp, payload value, alarm state, validation result, and downstream destination. The downstream system receives the message as validated clinical data rather than as an unverified radio packet.

Routing may be performed according to the context record, authorization record, gateway configuration, downstream-destination rule, packet priority, device type, patient location, session type, or governance policy. For example, routine patient-monitoring data may be routed to an electronic medical record system, high-frequency device data may be routed to a medical device data system or medical data governance system, device-status data may be routed to a biomed or hospital operations system, and alarm-associated data may be routed to a clinical command display or alerting system.

The authorized gateway may route the same validated source data to different downstream systems in different formats. For example, a representative value may be routed to an electronic medical record system, while higher-frequency data or waveform data is routed to a medical data governance system. A device-status message may be routed to a maintenance system, while patient-associated physiological data is routed to a clinical system. Each routed message may be associated with the same packet lifecycle record.

The system may suppress downstream routing when a duplicate packet is detected. A duplicate packet may be stored as duplicate-reception evidence, used for signal-quality or coverage analysis, or recorded in the audit trail without generating a second clinical message. The gateway may also suppress or flag delayed, stale, replay-suspect, or out-of-order packets according to downstream rules.

An audit trail is generated to preserve the packet lifecycle. The audit trail may include a packet receipt event, source authorization event, destination authorization event, encryption-key validation event, packet-sequence check, patient-context validation event, device-context validation event, location-context validation event, validation failure, quarantine action, decryption event, context-binding event, conversion event, downstream-routing event, duplicate-suppression event, delayed-packet reconciliation event, user confirmation event, correction event, and final release state.

Each audit event may include a timestamp, source-module identifier, device identifier, gateway identifier, packet sequence value, context reference, validation result, action taken, downstream destination, user identifier when applicable, and system component that performed the action. Audit events may be stored at the authorized gateway, medical device data system, electronic medical record system, medical data governance system, cloud system, or distributed across multiple systems.

In some embodiments, the audit trail records both rejected packets and released packets. A rejected or quarantined packet may have an audit trail showing why it was not released clinically. A released packet may have an audit trail showing how it was validated, decrypted, bound to context, converted, routed, and delivered. This permits later reconstruction of whether a LoRa-originated data point was associated with the correct patient, device, location, gateway, session, and downstream destination.

The audit trail may also record changes to context records. For example, if a clinician corrects a patient-device association, confirms a room change, updates a bed assignment, or approves delayed data, the correction may be linked to the affected packets and downstream messages. The system may then route corrected or reconciled messages while preserving the prior packet and validation history.

Downstream conversion and routing therefore occur only after the packet has passed the applicable validation and release-control steps. The conversion layer does not merely translate radio data into a clinical format. It converts validated, context-bound, and auditable LoRa-originated data into a form that downstream systems can safely process.

### Store-and-Forward, Data-Gap Detection, and Delayed Data Reconciliation

In some embodiments, the system supports store-and-forward operation when a LoRa communication path, relay node, authorized gateway, downstream system, or clinical network is unavailable, degraded, congested, or intermittently reachable. Store-and-forward operation may be performed by the LoRa communication module, one or more LoRa relay nodes, the authorized gateway, a medical device data system, a medical data governance system, or a distributed combination of these components.

When a packet cannot be immediately delivered, the packet may be stored with its packet sequence value, original event timestamp, source-module identifier, device identifier, context reference, packet priority, and retry state. The packet may remain encrypted while stored. Intermediate relay nodes do not need to decrypt the payload or read protected clinical-context metadata in order to store and later forward the packet.

The system may distinguish between the time at which source data was generated and the time at which the packet was received. The original event timestamp may identify when the physiological value, alarm, device-status value, or other source data was created. The received timestamp may identify when the packet reached a relay node, authorized gateway, downstream system, or medical data governance system. Preserving both timestamps allows delayed data to be reconciled without misrepresenting it as real-time data.

A retry scheduler may control later transmission of stored packets. The retry scheduler may adjust retry frequency, packet priority, hop count, time-to-live value, broadcast behavior, unicast behavior, or selected relay path based on packet urgency, gateway availability, signal quality, route availability, battery state, network congestion, or clinical classification. Alarm-associated or emergency packets may be retried more aggressively than routine packets.

When communication is restored, stored packets may be forwarded to the authorized gateway. The authorized gateway validates the packets against authorization and context records before clinical release. If the packet is valid but delayed, the gateway may mark the downstream message with a delay indicator, preserve the original event timestamp, suppress real-time alerting, or route the message for delayed reconciliation.

The system may detect duplicate packets. Duplicate detection may be based on packet sequence value, source-module identifier, device identifier, packet signature, original event timestamp, received timestamp, or context reference. Duplicate packets may be suppressed from downstream clinical routing while being stored as duplicate-reception evidence or used for relay-path, signal-quality, or gateway-coverage analysis.

The system may also detect stale, replay-suspect, or out-of-order packets. A stale packet may be too old for real-time clinical use. A replay-suspect packet may have a repeated sequence value, invalid timestamp relationship, invalid signature state, or other condition suggesting improper retransmission. An out-of-order packet may arrive after later packets from the same source or session have already been received. These conditions may cause the gateway to flag, quarantine, suppress, reconcile, or route the packet with a warning.

In addition to packet-level store-and-forward, the system may detect a data gap in the underlying medical-device or patient-associated data stream. A data gap may occur when packets are lost, a relay path fails, a gateway is unavailable, a patient moves out of range, a wearable disconnects, or a device stores data locally during disconnection. A missing-sequence detector, gateway, medical device data system, or medical data governance system may identify the missing interval.

When a data gap is detected, the system may generate a backfill request. The backfill request may be sent to the LoRa communication module, host medical device, patient-associated medical device, wearable, patient phone, local buffer, or other source that may retain locally stored source-device data. The request may identify a missing sequence value, time interval, patient-context reference, device-context reference, session identifier, or original event timestamp range.

The locally stored source-device data may then be transmitted after reconnection. The transmitted data may be encrypted and packetized in the same manner as current data, or it may be transmitted as a backfill packet with a delayed or backfill classification. The authorized gateway validates the backfilled data against authorization and context records before downstream release.

A time-synchronization engine may align delayed or backfilled data with previously received data. The engine may use original event timestamps, received timestamps, device clocks, gateway clocks, packet sequence values, session identifiers, care-location records, or medical data governance time references. The result may be a reconciled data stream in which delayed or backfilled data is inserted at the correct clinical time.

A reconciled downstream message may include current data, delayed data, backfilled data, corrected data, or gap-repaired data. The message may include an indicator showing that the data was delayed, backfilled, corrected, duplicate-suppressed, or time-synchronized. This allows the downstream system to receive a clinically useful data stream without losing the audit distinction between real-time receipt and later reconciliation.

The system may generate a reconciliation audit entry for each delayed, duplicate, stale, replay-suspect, out-of-order, or backfilled packet. The audit entry may identify the packet, source module, device, patient-context record, device-context record, original event timestamp, received timestamp, sequence value, missing interval, backfill request, locally stored data source, time-synchronization action, downstream message, and final routing or suppression action.

Store-and-forward and reconciliation therefore provide continuity without weakening the gateway release-control model. A delayed or backfilled packet is not treated as clinically valid merely because it was eventually received. It is validated, time-aligned, labeled when appropriate, and audited before downstream clinical release.

### Bluetooth, BLE, and LoRa Range-Transition Embodiments

In some embodiments, the system supports both short-range local communication and long-range LoRa communication. The short-range communication may be Bluetooth, Bluetooth Low Energy, a local proxy network, a bedside device connection, a phone connection, a local gateway connection, or another communication path available when the patient or device is within or near a care facility. LoRa communication may be used when the patient, device, or transport pathway is outside the reliable range of the short-range system.

A patient-associated medical device may first communicate with a nearby phone, bedside unit, local proxy node, wearable bridge, or LoRa communication module using Bluetooth or Bluetooth Low Energy. The nearby device may obtain source data from the patient-associated medical device and either forward the data through the short-range network or generate an encrypted LoRa packet for long-range transmission.

Inside a hospital or other care facility, a short-range proxy network may include phones, tablets, computers, bedside gateways, smart beds, smart IV poles, monitors, or other devices configured to receive Bluetooth or Bluetooth Low Energy data from patient-associated devices. These proxy devices may forward the data to a medical device data system, electronic medical record system, medical data governance system, or authorized gateway when local coverage is available.

When local short-range coverage is lost, degraded, or unavailable, the system may transition to LoRa transmission. The LoRa communication module may generate an encrypted LoRa packet containing source data from the host medical device or patient-associated medical device. The packet may then be transmitted directly to an authorized gateway or through one or more LoRa relay nodes.

The transition may occur automatically. The LoRa communication module, patient phone, local proxy device, authorized gateway, or medical data governance system may determine that the short-range path is unavailable based on signal strength, connection state, timeout, packet loss, gateway loss, patient movement, location change, or absence of a local proxy node. In response, the system may switch to LoRa transmission without requiring the patient to manually reconnect the medical device.

When the patient or device returns within range of the short-range network, the system may transition back to the short-range path. The transition may be based on detection of a known Bluetooth or Bluetooth Low Energy proxy node, bedside gateway, room gateway, hospital gateway, or other local node. The LoRa path may remain active as a backup, may be reduced to a lower reporting rate, or may be disabled until short-range coverage is lost again.

The same patient, device, context, and session associations may be maintained across the transition. A packet or data stream transmitted by the short-range path and a packet transmitted by the LoRa path may reference the same patient-context record, device-context record, care-location record, session identifier, or gateway association. This allows the authorized gateway or medical data governance system to reconcile data across the different communication paths.

In some embodiments, the system prevents duplicate downstream clinical routing when both the short-range path and the LoRa path deliver the same data. Duplicate suppression may be based on packet sequence value, original event timestamp, source-module identifier, device identifier, patient-context reference, device-context reference, or another shared identifier. One path may be treated as the primary path and the other as a backup path, or both may be retained as redundant delivery evidence.

The range-transition function may also support patient transport. During movement through a hospital, hallway, elevator, ambulance bay, ambulance, imaging department, operating room, home-care environment, or remote-care setting, short-range coverage may be intermittent. The system may use the short-range path when available and the LoRa path when needed to preserve continuity of patient-associated data.

In some embodiments, a patient phone or wearable bridge includes both a Bluetooth or Bluetooth Low Energy interface and a LoRa interface. The phone or bridge may receive data from one or more wearables by Bluetooth or Bluetooth Low Energy and transmit encrypted LoRa packets when the hospital short-range proxy network is unavailable. When the phone or bridge returns to the facility, it may rejoin the local proxy network and update the applicable context record or gateway association.

The transition does not by itself authorize downstream clinical release. Regardless of whether data arrives through a short-range path, LoRa path, or both, the authorized gateway or medical data governance system validates the source, destination, patient context, device context, session, and applicable authorization before clinical release. Thus, communication continuity and clinical validity remain separate functions.

In some embodiments, the system stores data locally during the transition. If Bluetooth, BLE, LoRa, or gateway communication is interrupted, the LoRa communication module, patient phone, wearable, host medical device, or relay node may retain locally stored data. When a communication path is restored, the system may transmit stored data with original event timestamps, sequence values, and delay or backfill indicators for validation and reconciliation.

The short-range-to-LoRa transition allows the same medical-device data stream to continue across facility coverage, transport coverage, remote-care coverage, and temporary outage conditions. The system uses the available communication path for transport, while the authorized gateway controls whether the data may be released as clinically valid downstream data.

### Emergency Responder Monitoring Embodiments

The emergency responder embodiments described in this subsection are implementations of the same LoRa communication architecture described above, in which the host or patient-associated medical device may be replaced by responder equipment, the protected clinical-context metadata may be replaced or supplemented by protected responder or incident-context metadata, and the authorized gateway may be implemented as an authorized incident command gateway.

In some embodiments, the same packet, relay, validation, and audit architecture is applied to emergency responder monitoring. The responder embodiment may be used for firefighters, emergency medical technicians, police officers, hazmat workers, search-and-rescue workers, military medics, industrial safety workers, confined-space workers, disaster-response workers, or other responders operating in environments where conventional communication may be degraded, unavailable, unsafe, or unreliable.

A responder-carried LoRa module may be attached to or integrated with responder equipment. The responder equipment may include turnout gear, helmet, belt, harness, self-contained breathing apparatus, oxygen apparatus, responder bracelet, medical patch, wearable sensor, handheld device, vehicle equipment, rescue equipment, or other personal protective or operational equipment.

The responder-carried LoRa module may obtain responder-status data from one or more responder-status interfaces. These interfaces may include a self-contained breathing apparatus interface, oxygen-pressure sensor interface, motion sensor, fall sensor, temperature sensor, biometric sensor, environmental sensor, helmet sensor, turnout-gear sensor, responder equipment interface, or manual distress input. The manual distress input may be a button, switch, gesture input, voice input, pull cord, or other user-actuated Mayday or distress mechanism.

Responder-status data may include responder identity, team assignment, incident identifier, equipment identifier, oxygen pressure, remaining air time, oxygen flow, motion state, no-motion state, fall state, temperature exposure, vital-sign state, battery state, distress state, Mayday state, environmental condition, location data, or gateway association. The data may be collected continuously, periodically, on demand, upon threshold crossing, upon manual distress activation, or upon gateway or communication loss.

The responder-carried LoRa module generates an encrypted responder-status LoRa packet. The packet may include a relay-readable header, protected responder metadata, and an encrypted responder-status payload. The relay-readable header may include routing information, responder-module identifier, command-gateway identifier, packet sequence value, hop count, time-to-live value, packet priority, packet type, or checksum. The protected responder metadata may include a responder-context reference, incident identifier, team identifier, equipment identifier, location reference, encryption-key identifier, event timestamp, gateway reference, or downstream destination. The responder-status payload may include encrypted responder-status data.

The responder packet may be transmitted directly to an incident command gateway or through fixed anchor nodes, deployable anchor nodes, vehicle gateways, perimeter nodes, relay nodes, mesh nodes, repeaters, or store-and-forward nodes. These nodes may be placed in or around a building, floor, tunnel, industrial site, vehicle, staging area, command area, perimeter, rescue route, or other incident scene. The relay nodes forward the packet using relay-readable information and do not decrypt the responder-status payload or access protected responder metadata in readable form.

An incident command gateway receives the encrypted responder-status LoRa packet and validates it before display or downstream routing. Validation may include responder-module authorization, command-gateway authorization, encryption-key validation, packet-sequence validation, responder-context validation, incident validation, team-assignment validation, equipment-assignment validation, or location-context validation. If validation fails, the incident command gateway may prevent display or downstream routing, quarantine or separately log the packet, request confirmation, or generate a validation-failure audit record.

If validation succeeds, the incident command gateway decrypts the responder-status payload and binds the data to the applicable responder-context record and incident record. The gateway may then generate or update an incident command display showing responder location, movement path, last-known location, fall state, no-motion state, oxygen status, temperature exposure, vital-sign state, battery state, distress state, Mayday state, equipment status, team assignment, or incident assignment.

An emergency classifier may classify responder-status data as routine, urgent, or emergency. Emergency classification may be based on oxygen status, remaining air time, no-motion duration, fall state, temperature exposure, vital-sign threshold, manual distress input, Mayday input, battery state, environmental condition, gateway-loss condition, or a combination of such conditions. In one example, the responder-carried LoRa module classifies a packet as emergency data when oxygen status satisfies a low-oxygen threshold and motion data indicates a no-motion condition for a threshold time period.

Responsive to emergency classification, the responder-carried LoRa module may increase packet priority, increase retry frequency, change hop count or time-to-live value, transmit through multiple relay paths, or transmit toward multiple incident command gateways. The incident command gateway may display the packet with a higher alert state, audible or visual warning, responder location emphasis, Mayday state, or escalation indicator.

Responder location may be determined or estimated using received signal strength, time-of-arrival information, time-difference-of-arrival information, anchor proximity, gateway proximity, hop path, inertial data, barometric data, floor-plan data, building map data, LIDAR data, manual confirmation, or combinations thereof. Location output may include a two-dimensional coordinate, three-dimensional coordinate, floor level, room, zone, stairwell, route, last-known location, confidence value, or movement path.

The incident command display may include a map, floor plan, building model, LIDAR model, three-dimensional model, command dashboard, vehicle display, tablet display, mobile command interface, browser interface, or other visual interface. Different command users may view different subsets of responder-status data depending on role, incident assignment, team assignment, authorization, or governance rule.

The system may store a last-known location and last-known responder-status state when communication with the responder-carried LoRa module is interrupted. If communication is later restored, delayed packets or locally stored responder-status data may be reconciled using packet sequence values, original event timestamps, received timestamps, incident records, responder-context records, and audit entries.

A responder packet lifecycle audit record may be generated. The audit record may link the responder packet, responder-carried LoRa module, responder identifier, responder-context record, incident record, incident command gateway, validation result, emergency classification, displayed responder status, location determination, downstream routing action, alert state, and timestamp. The audit record allows later reconstruction of responder-status receipt, validation, decryption, classification, display, routing, quarantine, correction, or escalation.

The responder embodiment therefore uses the LoRa relay infrastructure as a transport layer while preserving responder identity, incident context, and responder-status payload confidentiality. The incident command gateway controls whether received responder-status data is valid for display, routing, command action, or audit.

### Alternative Embodiments, Implementation Variations, and Non-Limiting Combinations

The embodiments described herein are examples and may be combined, separated, substituted, or modified without departing from the scope of the disclosure. A component described as being located in one device may be distributed among multiple devices. A function described as being performed by a LoRa communication module may be performed by an authorized gateway, medical device data system, medical data governance system, cloud system, local server, patient phone, relay node, or distributed combination thereof, where technically suitable.

The LoRa communication module may be implemented in different physical forms. Suitable forms include a dongle, embedded board, patch, label, chip, gateway card, wearable bridge, adapter, cartridge, plug-in module, replaceable insert, sealed module, clip-on module, adhesive module, or integrated radio component. The module may be installed during manufacture, added as a retrofit component, attached during patient admission, assigned during transport, or deployed during home-care or field operation.

The system is not limited to a particular LoRa implementation. The communication path may use LoRa, LoRaWAN, private LoRa, proprietary LoRa-compatible signaling, point-to-point LoRa, LoRa mesh, relay-assisted LoRa, anchor-assisted LoRa, gateway-directed LoRa, store-and-forward LoRa, or combinations thereof. The system may also be implemented with other long-range or low-power radio technologies, including Wi-Fi HaLow or another wireless technology capable of supporting the described packet, context, relay, gateway-validation, and release-control functions.

The system may operate with or without a public LoRaWAN network server, cellular network, Internet connection, carrier-operated infrastructure, or cloud service. In some embodiments, the communication path is a private path between a medical-device LoRa module, one or more relay nodes, and an authorized gateway controlled by a healthcare facility, home-care provider, emergency command system, medical device data system, medical data governance system, or other authorized entity.

The authorized gateway may be deployed as a room gateway, bed gateway, floor gateway, hospital gateway, vehicle gateway, ambulance gateway, home gateway, cloud gateway, incident command gateway, medical device data system gateway, medical data governance gateway, wall gateway, edge server, router-connected gateway, or software-defined gateway. Multiple gateways may receive the same packet. The system may deduplicate, reconcile, rank, or select among packet instances using packet sequence values, timestamps, signal quality, gateway priority, authorization records, context records, or governance rules.

The downstream system may be an electronic medical record system, medical device data system, medical data governance system, browser interface, clinical command system, hospital operations system, transport dashboard, biomed system, maintenance system, operating-room record system, cloud system, incident command system, responder-status dashboard, research system, or other authorized destination. Different downstream systems may receive different versions or subsets of the same validated source data.

The host device may be an infusion pump, ventilator, patient monitor, pulse oximeter, blood pressure monitor, glucose monitor, ECG monitor, capnography monitor, oxygen apparatus, smart bed, smart IV pole, wearable sensor, medical patch, patient phone, transport monitor, surgical device, imaging device, anesthesia device, oxygen concentrator, self-contained breathing apparatus, responder wearable, environmental sensor, or other medical, clinical, patient-associated, responder-associated, or operational device.

The local interface between the LoRa communication module and the host device may be wired, wireless, optical, mechanical, or logical. Suitable interfaces include serial, USB, Bluetooth, Bluetooth Low Energy, NFC-assisted pairing, RFID-assisted pairing, QR-assisted pairing, wired medical-device interface, proprietary medical-device port, sensor interface, optical interface, phone application interface, hospital application interface, or manual pairing input.

The system may be used in a hospital, clinic, emergency department, operating room, procedure room, imaging department, hallway, elevator, ambulance bay, ambulance, aircraft, ship, field hospital, long-term care facility, rehabilitation facility, home-care environment, rural clinic, remote-care site, disaster-response site, fireground, industrial site, military medical environment, or other environment where medical, device, patient, responder, location, or operational data must be transmitted with context control.

In some embodiments, edge processing may be performed before downstream routing. Edge processing may include threshold detection, alarm classification, event classification, packet prioritization, data reduction, duplicate detection, missing-sequence detection, gateway selection, context validation, signal-quality evaluation, route selection, battery-state evaluation, or local alert generation. Edge processing may be performed by the LoRa communication module, relay node, authorized gateway, incident command gateway, medical data governance system, or distributed combination thereof.

In some embodiments, bidirectional communication may be supported. The authorized gateway may transmit acknowledgements, provisioning data, route settings, key updates, firmware updates, sampling-rate settings, transmission-rate settings, packet-priority settings, pairing instructions, context updates, or configuration data to the LoRa communication module. Such commands may be limited by role authorization, device capability, manufacturer profile, safety rule, gateway authorization, encryption-key authorization, clinical context, or governance rule.

In some medical-device embodiments, bidirectional communication may be limited to communication-layer, provisioning-layer, routing-layer, alerting-layer, association-layer, security-layer, firmware-layer, audit-layer, or data-governance-layer functions. In other embodiments, a command that affects a therapeutic function, life-support function, drug-delivery function, ventilation function, oxygen-delivery function, or other treatment function of a host medical device may be permitted when separately authorized by host-device capability, manufacturer profile, user role, safety policy, clinical workflow, and an applicable governance rule.

The context records, authorization records, packet records, and audit records may be stored locally, at the gateway, at a medical device data system, at a medical data governance system, at an electronic medical record system, in a cloud system, or in a distributed architecture. A given packet may include a full context value, a context reference, a token, a key identifier, a pseudonymous identifier, or another value that allows the authorized gateway to retrieve and validate the applicable context before downstream release.

The described embodiments may be implemented using hardware, firmware, software, or combinations thereof. Processor-executable instructions may be stored in non-transitory memory at the LoRa communication module, authorized gateway, relay node, anchor node, medical device data system, medical data governance system, cloud system, incident command system, mobile device, patient phone, or other computing device. The instructions may cause the device to perform receiving, packetizing, encrypting, relaying, storing, forwarding, validating, decrypting, binding, converting, routing, quarantining, reconciling, displaying, alerting, auditing, or governing operations as described herein.

The order of operations described in this disclosure is illustrative. Operations may be performed in a different order, repeated, omitted, combined, divided, or performed by different components where technically suitable. For example, context validation may occur at an authorized gateway, medical data governance system, medical device data system, or distributed combination. Packet conversion may occur at the gateway or downstream interface engine. Audit records may be generated at multiple points and later reconciled.

The features described in the patient medical-device embodiments may be combined with features described in the emergency responder embodiments where technically appropriate. Likewise, store-and-forward, duplicate suppression, delayed-packet reconciliation, protected clinical-context metadata, relay-readable headers, encrypted payloads, gateway validation, context binding, and audit functions may be used separately or together in different combinations depending on the environment, device type, gateway configuration, and downstream clinical or command workflow.

### DETAILED DESCRIPTION OF THE FIGURES

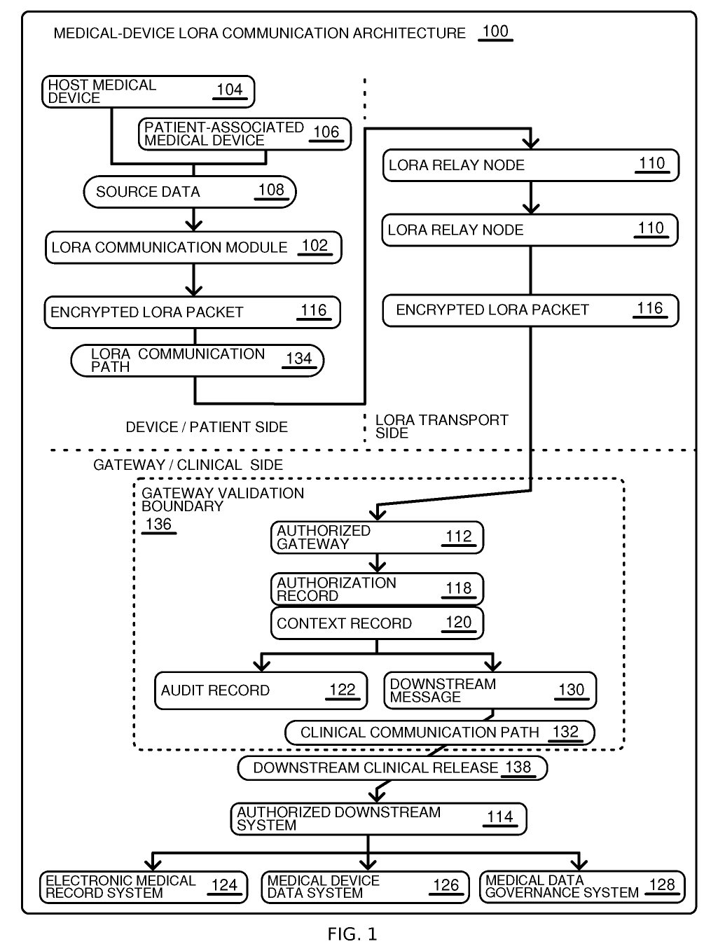

FIG. 1 — Overall Medical-Device LoRa Communication Architecture

FIG. 1 illustrates an example medical-device LoRa communication architecture 100 configured to transmit medical-device data, physiological data, alarm data, device-status data, device-identity data, or other patient-associated data from one or more medical devices or patient-associated devices to one or more authorized downstream clinical systems using LoRa or LoRa mesh communication.

The architecture 100 includes one or more LoRa communication modules 102 coupled to a host medical device 104 or a patient-associated medical device 106. The LoRa communication module 102 may be attached to, embedded in, plugged into, adhered to, clipped to, integrated with, or otherwise associated with the host medical device 104 or patient-associated medical device 106. In some embodiments, the LoRa communication module 102 may be implemented as a dongle, embedded module, patch, label, chip, gateway card, wearable bridge, plug-in module, or other communication component.

The LoRa communication module 102 obtains source data 108 from the host medical device 104 or patient-associated medical device 106. The source data 108 may include, without limitation, medical-device data, physiological data, alarm data, powered-state data, battery data, device-use data, device-identity data, device-context data, or other data associated with a patient, medical device, clinical event, care location, or monitoring session.

The LoRa communication module 102 generates an encrypted LoRa packet 116. In the illustrated embodiment, the encrypted LoRa packet 116 includes routing information usable by one or more LoRa relay nodes 110 and protected information usable by an authorized gateway 112. The encrypted LoRa packet 116 may include, as described in greater detail with respect to later figures, a relay header, protected clinical-context metadata, and an encrypted payload. The relay header permits routing or forwarding through the LoRa communication path 134, while the protected clinical-context metadata and encrypted payload preserve patient, device, care-location, and clinical data confidentiality during relay.

The LoRa relay nodes 110 are configured to receive the encrypted LoRa packet 116 and forward the encrypted LoRa packet 116 toward the authorized gateway 112 over the LoRa communication path 134. The LoRa relay nodes 110 may include relay nodes, anchor nodes, mesh nodes, repeaters, store-and-forward nodes, fixed nodes, mobile nodes, vehicle-mounted nodes, room nodes, bed-area nodes, transport-area nodes, or other LoRa-capable forwarding components. The LoRa relay nodes 110 may forward, repeat, retransmit, store, or route the encrypted LoRa packet 116 without decrypting the encrypted payload and without accessing protected clinical-context metadata in readable form. Accordingly, intermediate relay nodes may participate in long-range or mesh transmission while being prevented from reading patient-identifying, device-identifying, clinical-context, or payload information.

The authorized gateway 112 receives the encrypted LoRa packet 116 directly from the LoRa communication module 102 or indirectly through one or more LoRa relay nodes 110. The authorized gateway 112 includes or communicates with gateway processing and gateway memory resources configured to store or access one or more authorization records 118 and one or more context records 120. The context records 120 may include patient-context records, device-context records, room records, bed records, care-location records, gateway records, session records, downstream-destination records, or other records that preserve associations between received packet information and a patient, device, location, session, or authorized destination.

Before downstream clinical release 138 of decrypted payload data, the authorized gateway 112 validates packet information associated with the encrypted LoRa packet 116 against the authorization records 118 and the context records 120. Such validation may include determining whether the encrypted LoRa packet 116 corresponds to an authorized source, authorized destination, authorized communication path, valid patient context, valid device context, valid gateway association, valid care-location association, valid sequence value, or other stored authorization or context relationship. The gateway validation boundary 136 represents the logical boundary at which LoRa-originated data is checked before it may become clinically available data.

When validation is unsuccessful, the authorized gateway 112 prevents downstream clinical release 138 or downstream clinical routing of the encrypted LoRa packet 116. In some embodiments, the authorized gateway 112 may reject, quarantine, buffer, separately log, flag, suppress, or retain the encrypted LoRa packet 116 for later review, correction, reconciliation, or audit. Preventing downstream clinical release 138 avoids releasing data to a clinical system when the data cannot be validated as being associated with the correct patient, device, gateway, care location, or downstream destination.

When validation is successful, the authorized gateway 112 decrypts the encrypted payload, associates the decrypted payload data with the applicable context record 120, and makes the decrypted payload data available to one or more authorized downstream systems 114 through a clinical communication path 132. The authorized downstream systems 114 may include an electronic medical record system 124, a medical device data system 126, a medical data governance system 128, a browser interface, hospital operations system, transport dashboard, clinical command system, cloud system, or other authorized clinical, operational, or governance system.

In some embodiments, the authorized gateway 112 converts the validated and context-associated decrypted payload data into a downstream message 130. The downstream message 130 may be formatted as an HL7 message, FHIR resource, JSON object, XML object, MQTT message, HTTPS message, proprietary medical-device data message, EMR message, MDDS message, medical data governance message, or other downstream-compatible message format.

The authorized gateway 112 may further generate or update an audit record 122. The audit record 122 may link the encrypted LoRa packet 116, LoRa communication module 102, host medical device 104, patient-associated medical device 106, authorized gateway 112, authorization record 118, context record 120, validation result, downstream message 130, authorized downstream system 114, clinical communication path 132, LoRa communication path 134, gateway validation boundary 136, downstream clinical release 138, and timestamp. In this manner, the architecture 100 provides not merely wireless transport of medical data, but a governed medical-device LoRa communication architecture in which relay-readable routing information is separated from protected clinical-context information and encrypted payload data, and in which clinical downstream release is gated by authorization and patient/device-context validation.

In the embodiment of FIG. 1, the architecture 100 permits low-power, long-range, off-cellular-network, or private LoRa transmission of patient-associated or device-associated medical data while preserving privacy, context integrity, and downstream clinical reliability. The LoRa relay nodes 110 provide communication reach and path redundancy, while the authorized gateway 112 controls whether received LoRa-originated data may become clinically available data in an authorized downstream system 114.

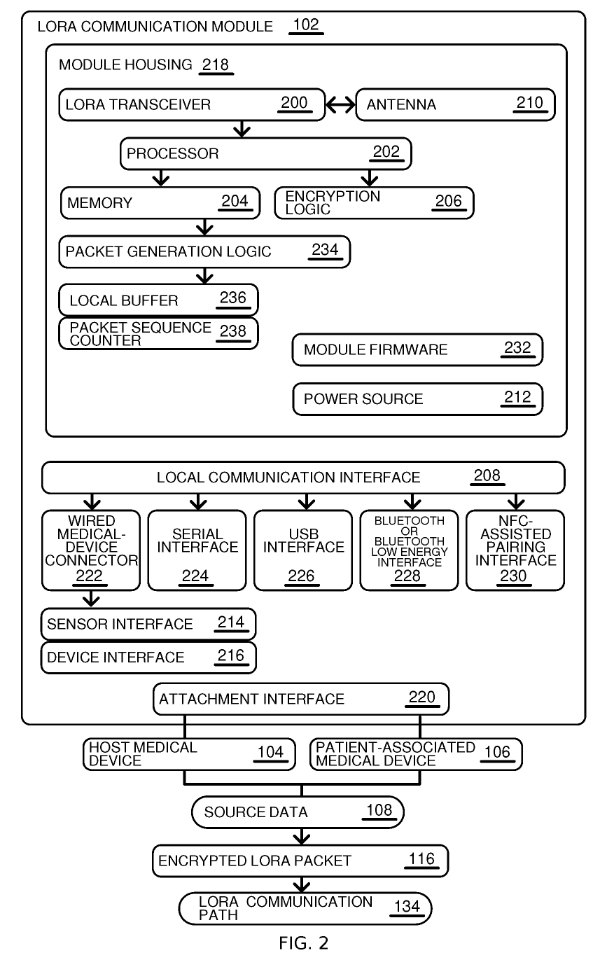

FIG. 2 — LoRa Communication Module / Dongle Hardware

FIG. 2 illustrates an example LoRa communication module 102 configured for coupling to a host medical device 104, patient-associated medical device 106, wearable sensor, patient phone, monitor, pump, ventilator, oxygen apparatus, smart bed, smart IV pole, transport monitor, clinical sensor, or other patient-associated or medical-device endpoint. The LoRa communication module 102 may be implemented as a dongle, patch, label, chip, embedded module, gateway card, wearable bridge, plug-in module, replaceable insert, cartridge, adapter, or other attachable or integrated communication component.

The LoRa communication module 102 includes a LoRa transceiver 200, a processor 202, memory 204, encryption logic 206, and a local communication interface 208. The LoRa transceiver 200 is configured to transmit and receive LoRa packets, including encrypted LoRa packets 116, over a LoRa communication path 134. The LoRa transceiver 200 may support direct point-to-point LoRa communication, private LoRa communication, LoRa mesh communication, relay-node communication, anchor-node communication, store-and-forward communication, gateway communication, or combinations thereof.

The processor 202 executes instructions stored in the memory 204 or otherwise accessible to the LoRa communication module 102. The processor 202 may perform packet generation, packet sequencing, source-data handling, encryption-key selection, protected-metadata generation, relay-header generation, local buffering, transmission scheduling, retransmission control, low-power operation, device-interface control, association handling, or other communication and data-governance functions. The processor 202 may be implemented as a microcontroller, microprocessor, system-on-chip, programmable logic device, digital signal processor, embedded controller, or other suitable processing component.

The memory 204 may store or receive identifiers, context references, security information, firmware, buffered packets, sequence values, device-interface profiles, and local configuration information. In some embodiments, the memory 204 stores or receives a source-module identifier, device identifier, patient-context reference, device-context reference, encryption-key identifier, destination-gateway identifier, packet sequence counter, routing parameter, packet priority, local device profile, or downstream-destination setting. The memory 204 may include volatile memory, non-volatile memory, secure memory, flash memory, electrically erasable memory, removable memory, or a distributed combination thereof.

The encryption logic 206 is configured to encrypt payload data, generate or apply encryption keys, select an encryption-key identifier, generate a packet signature, verify a key state, tokenize or protect context metadata, or otherwise protect medical-device data, physiological data, alarm data, device-status data, device-identity data, patient-context information, or device-context information before LoRa transmission. The encryption logic 206 may be implemented in hardware, firmware, software, secure memory, cryptographic circuitry, processor-executable instructions, or a combination thereof. In some embodiments, the encryption logic 206 causes the LoRa communication module 102 to generate the encrypted LoRa packet 116 having relay-readable information and gateway-protected information.

The local communication interface 208 provides communication between the LoRa communication module 102 and the host medical device 104, patient-associated medical device 106, or another local source of source data 108. The local communication interface 208 may include or communicate with a sensor interface 214, a device interface 216, a wired medical-device connector 222, a serial interface 224, a USB interface 226, a Bluetooth or Bluetooth Low Energy interface 228, an NFC-assisted pairing interface 230, an optical interface, proprietary device interface, wireless interface, contact interface, or another local communication pathway. Through the local communication interface 208, the LoRa communication module 102 obtains medical-device data, physiological data, alarm data, powered-state data, battery data, device-use data, device-identity data, device-context data, or other source data 108.

The LoRa communication module 102 may include an antenna 210 coupled to the LoRa transceiver 200. The antenna 210 may be internal, external, embedded, printed, flexible, detachable, shielded, directional, omnidirectional, or otherwise configured for transmission and reception of LoRa signals. In some embodiments, the antenna 210 is positioned or shaped based on the physical form factor of the module housing 218, the host medical device 104, the patient-associated medical device 106, the operating environment, a room layout, a transport workflow, or a responder or clinical use environment.

The LoRa communication module 102 may include a power source 212. The power source 212 may be an internal battery, rechargeable battery, replaceable battery, power cartridge, host-device power input, USB power input, vehicle power input, wall power input, gateway-supplied power input, energy-harvesting source, fire-safe power source, intrinsically safe power source, or other power supply. In some embodiments, the LoRa communication module 102 may draw power from the host medical device 104 when host power is available and transition to an internal or replaceable power source when host power is unavailable, interrupted, insufficient, or otherwise unsuitable.

The module housing 218 may enclose or support the LoRa transceiver 200, processor 202, memory 204, encryption logic 206, local communication interface 208, antenna 210, power source 212, sensor interface 214, device interface 216, and associated circuitry. The module housing 218 may be sealed, sterilizable, disposable, reusable, tamper-resistant, tamper-evident, waterproof, cleanable, biocompatible, fire-resistant, impact-resistant, intrinsically safe, or otherwise configured for a medical, clinical, transport, home-care, emergency-response, or hazardous operating environment.

An attachment interface 220 allows the LoRa communication module 102 to be attached to, embedded in, plugged into, adhered to, clipped to, mounted on, inserted into, coupled with, or otherwise associated with the host medical device 104 or patient-associated medical device 106. The attachment interface 220 may include an adhesive surface, clip, bracket, plug, connector, housing slot, battery-compartment insert, cable interface, lanyard, strap, snap-fit feature, magnetic coupling, screw mount, rail mount, sterile cover interface, or other mechanical or electrical coupling arrangement.

The sensor interface 214 may receive signals from one or more sensors associated with the host medical device 104, patient-associated medical device 106, patient, care location, or operating environment. The sensor interface 214 may receive physiological signals, environmental signals, motion signals, pressure signals, oxygen signals, temperature signals, location signals, alarm signals, powered-state signals, battery signals, or other sensor-generated information. The device interface 216 may receive device data, device identity, device status, alarm state, operating state, powered state, device-use state, maintenance state, calibration state, serial number, regulated-device identifier, firmware version, or other device-associated information.

The wired medical-device connector 222, serial interface 224, USB interface 226, Bluetooth or Bluetooth Low Energy interface 228, and NFC-assisted pairing interface 230 are examples of local interfaces that may be used individually or in combination. The wired medical-device connector 222 may connect to a proprietary medical-device port, monitoring port, accessory port, service port, or device-data port. The serial interface 224 may include RS-232, RS-485, UART, or another serial communication path. The USB interface 226 may provide data communication, power, or both. The Bluetooth or Bluetooth Low Energy interface 228 may receive data from a wearable, phone, monitor, patch, sensor, or patient-associated device. The NFC-assisted pairing interface 230 may support pairing, provisioning, patient-device association, device-context association, or confirmation of a patient, device, room, bed, care-location, or gateway relationship.

The LoRa communication module 102 may execute module firmware 232. The module firmware 232 may include instructions for receiving source data 108, assigning packet sequence values, selecting encryption keys, generating encrypted payloads, generating relay headers, generating protected clinical-context metadata, transmitting encrypted LoRa packets 116, receiving acknowledgements, storing packets, entering low-power modes, retransmitting packets, updating configuration settings, or responding to provisioning instructions from an authorized gateway 112 or other authorized system.

Packet generation logic 234 may be implemented as part of the processor 202, encryption logic 206, module firmware 232, or a distributed combination thereof. The packet generation logic 234 is configured to generate the encrypted LoRa packet 116 by forming relay-readable routing information, protected clinical-context metadata, and encrypted payload data. The packet generation logic 234 may use one or more values stored in or received by the memory 204, including identifiers, context references, encryption-key identifiers, packet sequence values, packet priority, hop count, time-to-live value, gateway identifiers, or downstream-destination information.

The LoRa communication module 102 may include a local buffer 236. The local buffer 236 may temporarily store source data 108, encrypted LoRa packets 116, unsent packets, retransmission packets, delayed packets, packet fragments, acknowledgement states, sequence information, device-status information, or other data. In some embodiments, the local buffer 236 supports store-and-forward operation when a LoRa communication path 134, LoRa relay node 110, authorized gateway 112, or authorized downstream system 114 is unavailable.

The packet sequence counter 238 may be used to assign a packet sequence value to each encrypted LoRa packet 116 generated by the LoRa communication module 102. The packet sequence counter 238 may support duplicate-packet detection, delayed-packet detection, replay-suspect detection, out-of-order packet detection, missing-sequence detection, packet reconciliation, audit-record generation, or downstream clinical release control at the authorized gateway 112 or other downstream system.

In operation, the LoRa communication module 102 obtains source data 108 through the local communication interface 208, applies encryption and context-protection functions using the encryption logic 206, generates an encrypted LoRa packet 116 using the packet generation logic 234, and transmits the encrypted LoRa packet 116 through the LoRa transceiver 200 and antenna 210. The encrypted LoRa packet 116 may be transmitted directly to the authorized gateway 112 or indirectly through one or more LoRa relay nodes 110. In this manner, the LoRa communication module 102 converts a host medical device 104 or patient-associated medical device 106 into a LoRa-enabled, context-preserving, gateway-validated endpoint without requiring the intermediate LoRa relay nodes 110 to access protected clinical-context metadata or encrypted payload data in readable form.

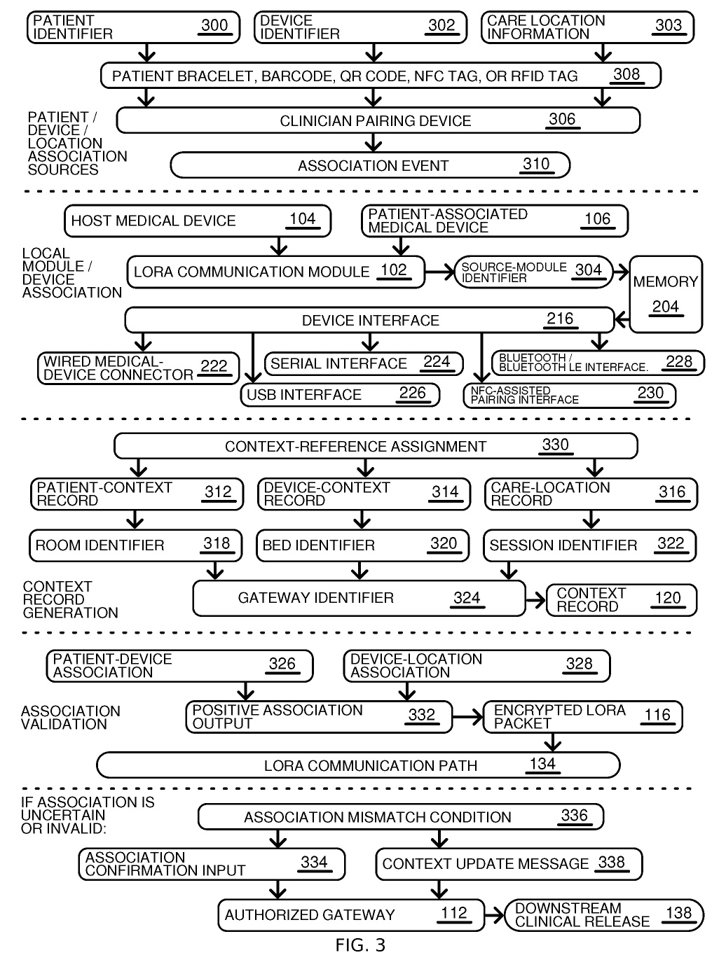

FIG. 3 — Local Interface and Patient/Device/Context Association

FIG. 3 illustrates an example local interface and patient/device/context association workflow in which a LoRa communication module 102 obtains source data 108 from a host medical device 104 or patient-associated medical device 106 and associates the source data 108 with one or more context records 120 before or during generation of an encrypted LoRa packet 116.

The workflow of FIG. 3 may be performed during patient admission, bedside device setup, patient monitoring, transport preparation, remote monitoring preparation, operating-room preparation, discharge preparation, emergency response staging, device replacement, device reassignment, or another clinical or operational workflow. The workflow may be performed by the LoRa communication module 102, an authorized gateway 112, a clinician pairing device 306, an electronic medical record system 124, a medical device data system 126, a medical data governance system 128, or a distributed combination thereof.

A patient identifier 300 may be obtained from a patient bracelet, barcode, QR code, NFC tag, RFID tag, biometric input, patient phone, hospital application, admission system, electronic medical record system 124, medical data governance system 128, clinician input, or another patient-identification source. The patient identifier 300 may be a medical record number, encounter identifier, admission identifier, tokenized patient identifier, pseudonymous patient identifier, session-specific patient reference, or other identifier suitable for associating medical-device data with a patient or patient context.

A device identifier 302 may be obtained from the host medical device 104, patient-associated medical device 106, LoRa communication module 102, device label, barcode, QR code, NFC tag, RFID tag, serial-number field, regulated-device identifier, manufacturer identifier, model identifier, software version, firmware version, equipment record, device interface 216, wired medical-device connector 222, serial interface 224, USB interface 226, Bluetooth or Bluetooth Low Energy interface 228, NFC-assisted pairing interface 230, or another device-identification source. The device identifier 302 may identify the host medical device 104, patient-associated medical device 106, LoRa communication module 102, or a particular device session.

Care-location information 303 may include information identifying or representing a room, bed, care area, department, transport route, gateway area, home-care location, field location, or other physical or logical location associated with a patient, device, module, session, or care event.

A source-module identifier 304 identifies the LoRa communication module 102 that obtains source data 108 or generates the encrypted LoRa packet 116. The source-module identifier 304 may be stored in the memory 204 of the LoRa communication module 102, provisioned during device setup, assigned by the authorized gateway 112, derived from a cryptographic credential, associated with an encryption-key identifier, or linked to a context record 120. The source-module identifier 304 may be used later by the authorized gateway 112 to validate whether the encrypted LoRa packet 116 originated from an authorized source.

The clinician pairing device 306 may include a smartphone, tablet, scanner, workstation, handheld reader, mobile computer, wearable computer, or another device used by a clinician, technician, caregiver, responder, biomedical engineer, or authorized user to confirm or initiate an association event 310. The clinician pairing device 306 may read a patient bracelet, barcode, QR code, NFC tag, RFID tag, or other association artifact 308 to obtain the patient identifier 300, device identifier 302, room identifier 318, bed identifier 320, gateway identifier 324, or other association data.

The association artifact 308 may be located on or near the patient, host medical device 104, patient-associated medical device 106, bed, room, wall, transport device, wearable sensor, patient phone, equipment item, gateway, care-location marker, or another object associated with the clinical workflow. In some embodiments, the association artifact 308 is reachable even when the host medical device 104 or patient-associated medical device 106 is physically inaccessible, mounted behind equipment, located above a bed, enclosed in a housing, positioned in a sterile field, or otherwise difficult to reach directly.

The association event 310 links one or more identifiers or references to form or update the context record 120. The association event 310 may be triggered by scanning, tapping, pairing, clinician confirmation, automatic proximity detection, Bluetooth detection, LoRa detection, NFC detection, QR-code scanning, RFID scanning, admission/discharge/transfer data, electronic medical record data, gateway detection, device-interface data, or another association action.

The context record 120 may include or reference a patient-context record 312, a device-context record 314, and a care-location record 316. The patient-context record 312 may link the patient identifier 300 to an encounter, admission, session, care event, room, bed, device, gateway, or downstream destination. The device-context record 314 may link the device identifier 302 or source-module identifier 304 to a host medical device 104, patient-associated medical device 106, device type, regulated-device identifier, serial number, firmware version, powered state, alarm state, maintenance state, calibration state, assigned patient, assigned room, assigned bed, or authorized gateway. The care-location record 316 may link the patient, device, module, or packet to a care location, room, bed, floor, department, transport route, procedure area, operating room, imaging area, emergency department, ambulance bay, home-care location, or other location context.

The room identifier 318 and bed identifier 320 may identify a physical or logical care location associated with the patient identifier 300, device identifier 302, source-module identifier 304, or session identifier 322. The room identifier 318 and bed identifier 320 may be obtained from a hospital admission/discharge/transfer system, electronic medical record system 124, medical data governance system 128, gateway proximity, wall-mounted marker, bedside marker, clinician pairing device 306, association artifact 308, local wireless detection, or another source. The session identifier 322 may identify a monitoring session, care event, transport event, procedure, incident, device-use interval, or other time-bounded association.

The gateway identifier 324 identifies an authorized gateway 112 or gateway association that may receive the encrypted LoRa packet 116 or validate the encrypted LoRa packet 116 before downstream clinical release 138. The gateway identifier 324 may correspond to a room gateway, bed gateway, floor gateway, hospital gateway, vehicle gateway, home gateway, incident command gateway, medical device data system gateway, medical data governance gateway, or other authorized gateway 112.

A patient-device association 326 links the patient identifier 300 with the device identifier 302, source-module identifier 304, host medical device 104, patient-associated medical device 106, or LoRa communication module 102. The patient-device association 326 may be created before data collection, during data collection, after data collection, or during later reconciliation. In some embodiments, the patient-device association 326 must be validated before decrypted payload data from the encrypted LoRa packet 116 may be released to an authorized downstream system 114.

A device-location association 328 links the device identifier 302, source-module identifier 304, host medical device 104, patient-associated medical device 106, or LoRa communication module 102 to a room identifier 318, bed identifier 320, gateway identifier 324, or care-location record 316. The device-location association 328 may be used to detect a room mismatch, bed mismatch, care-location mismatch, gateway mismatch, patient-device mismatch, expired association, or other association inconsistency.

A context-reference assignment 330 assigns or generates a patient-context reference, device-context reference, care-location reference, session reference, gateway reference, or downstream-destination reference for inclusion in or association with the encrypted LoRa packet 116. In some embodiments, the context-reference assignment 330 generates protected clinical-context metadata later carried in the encrypted LoRa packet 116. In other embodiments, the encrypted LoRa packet 116 includes a token, pointer, key, or identifier that allows the authorized gateway 112 to retrieve the applicable context record 120.

A positive association output 332 may be generated when the patient identifier 300, device identifier 302, source-module identifier 304, room identifier 318, bed identifier 320, session identifier 322, gateway identifier 324, patient-device association 326, and device-location association 328 satisfy one or more association rules. The positive association output 332 may authorize packet generation, packet transmission, gateway validation, decryption, downstream routing, or downstream clinical release 138.

An association confirmation input 334 may be provided by a clinician, technician, caregiver, patient, responder, biomedical engineer, authorized user, electronic medical record system 124, medical data governance system 128, or another system. The association confirmation input 334 may confirm or correct a patient-device association 326, device-location association 328, context-reference assignment 330, session identifier 322, room identifier 318, bed identifier 320, or gateway identifier 324.

An association mismatch condition 336 may occur when the patient identifier 300, device identifier 302, source-module identifier 304, room identifier 318, bed identifier 320, session identifier 322, gateway identifier 324, patient-device association 326, or device-location association 328 is missing, inconsistent, expired, unauthorized, duplicated, mismatched, or otherwise invalid. When an association mismatch condition 336 is detected, the system may prevent downstream clinical release 138, request re-pairing, generate an alert, quarantine a packet, separately log the packet, update an audit record 122, or require an association confirmation input 334.

A context update message 338 may be generated to update the context record 120, patient-context record 312, device-context record 314, care-location record 316, authorization record 118, authorized gateway 112, authorized downstream system 114, electronic medical record system 124, medical device data system 126, or medical data governance system 128. The context update message 338 may reflect a new association, corrected association, expired association, transport event, room change, bed change, device replacement, module replacement, patient discharge, session termination, or other change in clinical context.

In operation, the workflow of FIG. 3 permits source data 108 obtained by the LoRa communication module 102 to be associated with the correct patient, device, care location, gateway, and session before the source data 108 is released downstream as clinical data. The workflow also supports deferred or corrected association, in which source data 108 may be collected before a final patient-device association 326 or device-location association 328 is confirmed, provided that downstream clinical release 138 is prevented until the authorized gateway 112 or another authorized system validates the applicable context record 120.

The association workflow of FIG. 3 therefore provides the clinical-context foundation for the encrypted LoRa packet 116 and for gateway-side validation. The LoRa communication module 102 can transmit data through a LoRa communication path 134, while the authorized gateway 112 can prevent downstream clinical release 138 unless the packet information corresponds to a valid patient-context record 312, device-context record 314, care-location record 316, authorization record 118, or other stored clinical-context relationship.

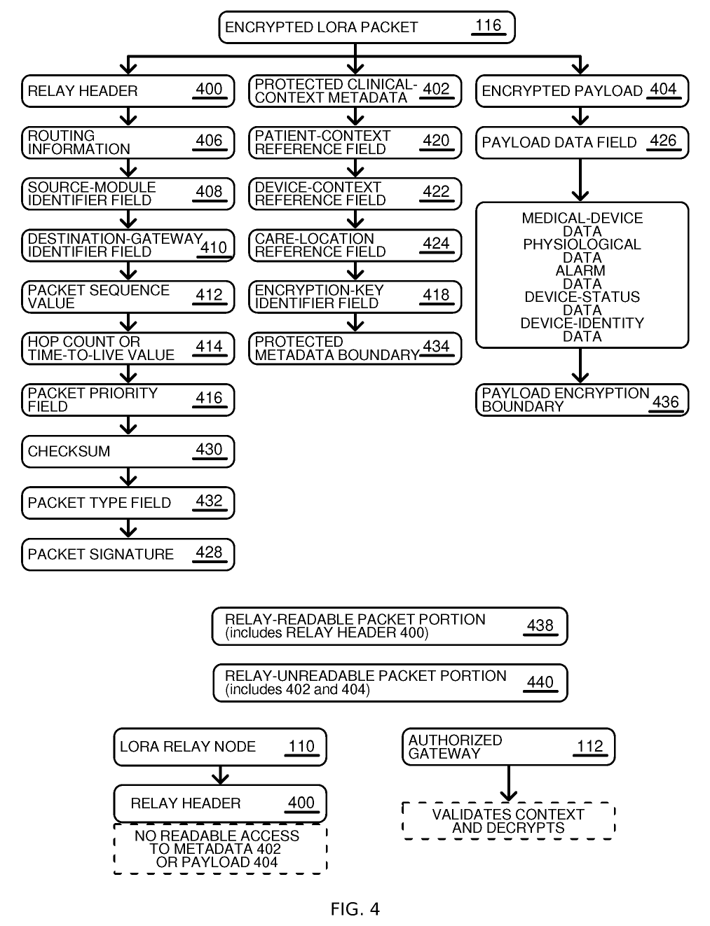

FIG. 4 — Three-Part Encrypted LoRa Packet Structure

FIG. 4 illustrates an example encrypted LoRa packet 116 generated by the LoRa communication module 102 for transmission over the LoRa communication path 134. The encrypted LoRa packet 116 is configured to permit one or more LoRa relay nodes 110 to forward the encrypted LoRa packet 116 toward the authorized gateway 112 while preventing the LoRa relay nodes 110 from accessing protected clinical-context information or medical-device payload information in readable form.

In the illustrated embodiment, the encrypted LoRa packet 116 includes a relay header 400, protected clinical-context metadata 402, and an encrypted payload 404. The relay header 400 is a relay-readable portion of the encrypted LoRa packet 116. The protected clinical-context metadata 402 is a gateway-protected portion of the encrypted LoRa packet 116. The encrypted payload 404 is a payload portion containing encrypted source data 108, such as medical-device data, physiological data, alarm data, device-status data, powered-state data, battery data, device-use data, device-identity data, or other patient-associated data.

The relay header 400 includes routing information 406 that permits the LoRa relay nodes 110 to determine whether and how to forward the encrypted LoRa packet 116 toward the authorized gateway 112. The routing information 406 may include one or more of a source-module identifier field 408, destination-gateway identifier field 410, packet sequence value 412, hop count or time-to-live value 414, packet priority field 416, packet type field 432, checksum 430, routing parameter, relay class, packet-size indicator, or other relay-readable value. The relay header 400 may be readable by the LoRa relay nodes 110 without allowing the LoRa relay nodes 110 to read the protected clinical-context metadata 402 or encrypted payload 404 in readable form.

The source-module identifier field 408 may identify the LoRa communication module 102 that generated the encrypted LoRa packet 116. The destination-gateway identifier field 410 may identify the authorized gateway 112 or class of authorized gateways intended to receive or validate the encrypted LoRa packet 116. The packet sequence value 412 may be assigned using the packet sequence counter 238 and may be used for duplicate-packet detection, delayed-packet detection, replay-suspect detection, out-of-order detection, missing-sequence detection, gateway validation, packet reconciliation, or audit-record generation.

The hop count or time-to-live value 414 may define whether the encrypted LoRa packet 116 may continue to be forwarded by one or more LoRa relay nodes 110. In some embodiments, the hop count or time-to-live value 414 is decremented, updated, or otherwise modified by a LoRa relay node 110 before retransmission. The packet priority field 416 may indicate whether the encrypted LoRa packet 116 is routine, urgent, emergency, alarm-associated, delayed, retransmitted, store-and-forward, or otherwise prioritized for relay or gateway handling.

The encryption-key identifier field 418 may identify an encryption key, session key, gateway key, device key, patient-context key, module key, responder key, rotating key, or other cryptographic reference used to encrypt, decrypt, validate, or otherwise process the encrypted LoRa packet 116. In some embodiments, the encryption-key identifier field 418 is included in the protected clinical-context metadata 402. In other embodiments, the encryption-key identifier field 418 may be included in the relay header 400, in a gateway-readable portion of the packet, or in both a relay-readable and a protected portion depending on the security configuration. When the encryption-key identifier field 418 is protected, LoRa relay nodes 110 may forward the encrypted LoRa packet 116 without learning the key association used for clinical-context validation or payload decryption.

The protected clinical-context metadata 402 includes information that associates the encrypted LoRa packet 116 with one or more context records 120. The protected clinical-context metadata 402 may include a patient-context reference field 420, device-context reference field 422, care-location reference field 424, encryption-key identifier field 418, gateway identifier, session identifier, room identifier, bed identifier, procedure identifier, downstream-destination identifier, access-control rule identifier, routing-rule identifier, quarantine-rule identifier, audit-rule identifier, timestamp, event timestamp, or other clinical-context information. The protected clinical-context metadata 402 may be encrypted, signed, tokenized, pseudonymized, gateway-readable only, or otherwise inaccessible in readable form to the LoRa relay nodes 110.

The patient-context reference field 420 may reference a patient-context record 312 associated with a patient identifier 300, encounter, admission, session, room, bed, device, gateway, care location, or downstream destination. The device-context reference field 422 may reference a device-context record 314 associated with a device identifier 302, source-module identifier 304, host medical device 104, patient-associated medical device 106, device type, serial number, regulated-device identifier, device state, alarm state, maintenance state, calibration state, or assigned patient. The care-location reference field 424 may reference a care-location record 316 associated with a room identifier 318, bed identifier 320, gateway identifier 324, transport route, care area, department, or other location context.

The encrypted payload 404 includes a payload data field 426. The payload data field 426 contains encrypted source data 108 obtained from the host medical device 104 or patient-associated medical device 106. The payload data field 426 may include medical-device data, physiological data, alarm data, device-status data, powered-state data, battery data, device-use data, device-identity data, waveform data, vital-sign data, infusion data, ventilation data, pulse oximetry data, ECG data, blood pressure data, glucose data, temperature data, capnography data, oxygen data, patient-input data, or other patient-associated data.

The encrypted LoRa packet 116 may further include a packet signature 428 and checksum 430. The packet signature 428 may be used by the authorized gateway 112 to determine whether the encrypted LoRa packet 116 originated from an authorized source, has been altered, has an expected key relationship, or corresponds to a valid authorization record 118. The checksum 430 may be used to detect transmission errors, packet corruption, packet truncation, or other communication integrity problems. In some embodiments, the checksum 430 is relay-readable so that LoRa relay nodes 110 may discard corrupted packets without reading protected clinical-context metadata 402 or encrypted payload 404.

The packet type field 432 may identify the encrypted LoRa packet 116 as a routine data packet, alarm packet, emergency packet, acknowledgement packet, retransmission packet, store-and-forward packet, delayed packet, configuration packet, association packet, context-update packet, gateway-directed packet, or other packet type. The packet type field 432 may be relay-readable, gateway-readable, protected, or distributed across the relay header 400 and protected clinical-context metadata 402.

The protected metadata boundary 434 represents a logical, cryptographic, tokenization, signing, or access-control boundary between relay-readable packet information and protected clinical-context information. The payload encryption boundary 436 represents a logical or cryptographic boundary around the encrypted payload 404. The relay-readable packet portion 438 includes the relay header 400 and any packet information that the LoRa relay nodes 110 may use to forward, store, retransmit, prioritize, or discard the encrypted LoRa packet 116. The relay-unreadable packet portion 440 includes the protected clinical-context metadata 402 and encrypted payload 404, or portions thereof, that are not accessible in readable form to the LoRa relay nodes 110.

By separating the encrypted LoRa packet 116 into the relay header 400, protected clinical-context metadata 402, and encrypted payload 404, the system permits LoRa relay nodes 110 to perform routing, forwarding, hop-count handling, packet-priority handling, checksum handling, retransmission, or store-and-forward operations without access to patient-identifying, device-identifying, clinical-context, or medical payload information. The authorized gateway 112 can then validate the packet using authorization records 118 and context records 120 before downstream clinical release 138.

In operation, the LoRa communication module 102 obtains source data 108, generates the encrypted payload 404, generates or attaches the protected clinical-context metadata 402, generates the relay header 400, and transmits the encrypted LoRa packet 116 through the LoRa communication path 134. A LoRa relay node 110 reads the relay header 400 or relay-readable packet portion 438, forwards the encrypted LoRa packet 116, and does not access the relay-unreadable packet portion 440 in readable form. The authorized gateway 112 receives the encrypted LoRa packet 116, validates packet information and protected clinical-context information against one or more authorization records 118 and context records 120, decrypts the encrypted payload 404 when validation is successful, and prevents downstream clinical release 138 when validation is unsuccessful.

The packet structure of FIG. 4 therefore provides a technical separation between communication-layer routing and clinical-context access. This separation allows untrusted, semi-trusted, mobile, temporary, or non-clinical LoRa relay nodes 110 to participate in the LoRa communication path 134 while maintaining clinical privacy, patient/device-context integrity, and authorized-gateway control over downstream clinical release 138.

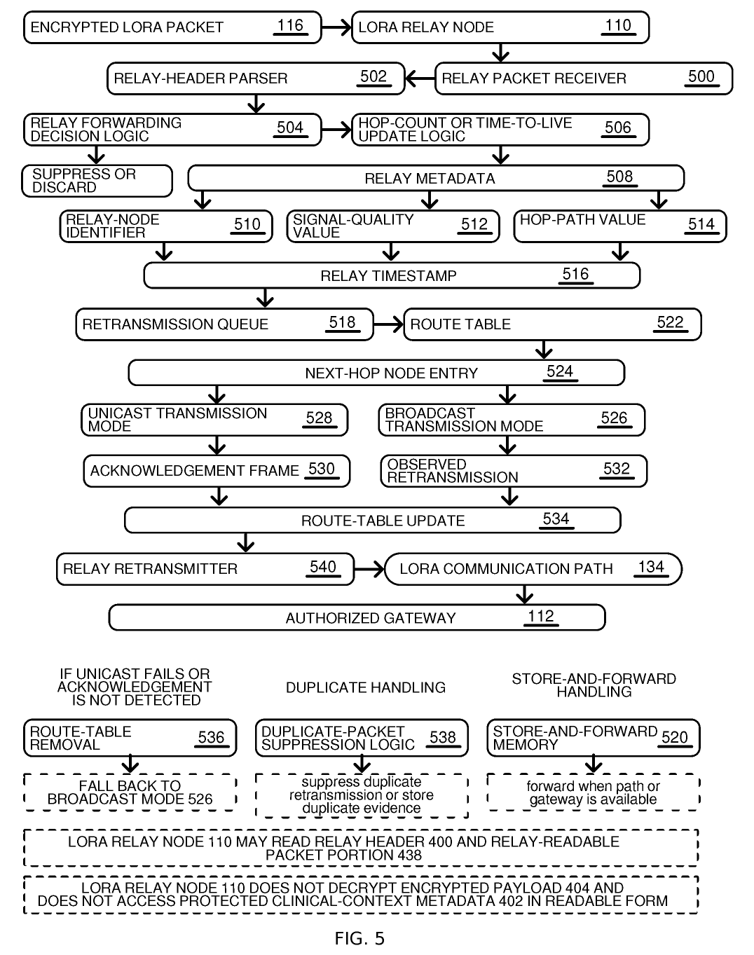

FIG. 5 — Relay-Node Forwarding Without Payload or Metadata Access

FIG. 5 illustrates an example relay-node forwarding workflow in which one or more LoRa relay nodes 110 receive an encrypted LoRa packet 116 and forward the encrypted LoRa packet 116 toward an authorized gateway 112 over a LoRa communication path 134. The workflow of FIG. 5 permits LoRa relay nodes 110 to participate in long-range, mesh, private, store-and-forward, or intermittent communication while preventing the LoRa relay nodes 110 from decrypting the encrypted payload 404 or accessing protected clinical-context metadata 402 in readable form.

A LoRa relay node 110 includes or communicates with a relay packet receiver 500. The relay packet receiver 500 receives the encrypted LoRa packet 116 from a LoRa communication module 102, another LoRa relay node 110, a fixed anchor node, deployable anchor node, vehicle-mounted relay, room relay, bed-area relay, transport relay, gateway-adjacent relay, or other LoRa-capable node. The relay packet receiver 500 may receive the encrypted LoRa packet 116 during direct transmission, broadcast transmission, unicast transmission, retransmission, delayed forwarding, route discovery, or store-and-forward operation.

After receiving the encrypted LoRa packet 116, the LoRa relay node 110 uses a relay-header parser 502 to read the relay header 400 or relay-readable packet portion 438. The relay-header parser 502 may read routing information 406, source-module identifier field 408, destination-gateway identifier field 410, packet sequence value 412, hop count or time-to-live value 414, packet priority field 416, packet type field 432, checksum 430, or other relay-readable information. The relay-header parser 502 does not decrypt the encrypted payload 404 and does not access the protected clinical-context metadata 402 in readable form.

Relay forwarding decision logic 504 determines whether the encrypted LoRa packet 116 should be forwarded, stored, retransmitted, ignored, suppressed, or discarded. The relay forwarding decision logic 504 may evaluate the hop count or time-to-live value 414, packet priority field 416, destination-gateway identifier field 410, packet sequence value 412, checksum 430, duplicate-packet state, route-table state, signal-quality state, congestion state, node power state, or other relay-readable information. If the hop count or time-to-live value 414 does not permit further forwarding, the relay forwarding decision logic 504 may suppress or discard the encrypted LoRa packet 116 without accessing the relay-unreadable packet portion 440.

Hop-count or time-to-live update logic 506 may update the hop count or time-to-live value 414 before retransmission. For example, the hop-count or time-to-live update logic 506 may decrement a hop count, reduce a time-to-live value, mark a packet as relayed, update a relay count, or otherwise modify relay-header information while leaving the protected clinical-context metadata 402 and encrypted payload 404 unchanged and unreadable to the LoRa relay node 110.

The LoRa relay node 110 may append relay metadata 508 to the encrypted LoRa packet 116 or to a relay event record associated with the encrypted LoRa packet 116. The relay metadata 508 may include a relay-node identifier 510, signal-quality value 512, hop-path value 514, relay timestamp 516, store-and-forward state, retry counter, received-power indication, link-quality indicator, node battery state, queue state, route-table state, or another relay-observable value. In some embodiments, relay metadata 508 is appended to the relay header 400 or to a relay-readable extension. In other embodiments, relay metadata 508 is stored locally or transmitted separately to the authorized gateway 112.

The relay-node identifier 510 may identify the LoRa relay node 110 that received, stored, forwarded, or retransmitted the encrypted LoRa packet 116. The signal-quality value 512 may represent received signal strength, link quality, signal-to-noise ratio, time-of-arrival information, proximity, gateway confidence, or another communication quality value. The hop-path value 514 may represent one or more previous or next relay nodes, a path index, route class, relay chain, next-hop node, previous-hop node, or other path-related information. The relay timestamp 516 may identify when the encrypted LoRa packet 116 was received, stored, forwarded, retransmitted, or suppressed by the LoRa relay node 110.

A retransmission queue 518 may temporarily hold encrypted LoRa packets 116 awaiting transmission. The retransmission queue 518 may be ordered based on packet priority, time received, hop count, destination-gateway identifier, route-table state, signal-quality state, retry count, emergency status, alarm status, or other relay-readable information. A store-and-forward memory 520 may store encrypted LoRa packets 116 when the authorized gateway 112, next-hop node, LoRa communication path 134, or downstream communication path is unavailable, congested, obstructed, degraded, or intermittent. The retransmission queue 518 and store-and-forward memory 520 store the encrypted LoRa packet 116 without decrypting the encrypted payload 404 and without accessing protected clinical-context metadata 402 in readable form.

The LoRa relay node 110 may include or access a route table 522. The route table 522 may associate a destination, destination-gateway identifier field 410, authorized gateway 112, final node, route class, care area, gateway group, or other destination reference with a next-hop node entry 524. The next-hop node entry 524 may identify another LoRa relay node 110, LoRa communication module 102, anchor node, gateway, vehicle gateway, room gateway, bed gateway, or other LoRa-capable destination. The route table 522 may be used to select unicast transmission mode 528 when a next-hop node entry 524 is available.

When no next-hop node entry 524 is available, when a route-table entry is stale, when acknowledgement is not detected, when signal quality is insufficient, when a next-hop node is unavailable, or when a packet priority or route condition requires broader transmission, the LoRa relay node 110 may select broadcast transmission mode 526. In broadcast transmission mode 526, the encrypted LoRa packet 116 may be transmitted to multiple receiving nodes without exposing the encrypted payload 404 or protected clinical-context metadata 402 in readable form. In unicast transmission mode 528, the encrypted LoRa packet 116 may be transmitted to a selected next-hop node based on the route table 522 or next-hop node entry 524.

An acknowledgement frame 530 may be received by the LoRa relay node 110 from another LoRa relay node 110, the authorized gateway 112, or another node. The acknowledgement frame 530 may indicate that the encrypted LoRa packet 116 or a packet identified by the packet sequence value 412 was received, forwarded, accepted, or delivered. In some embodiments, the acknowledgement frame 530 includes relay-readable information sufficient to update the route table 522 without disclosing protected clinical-context metadata 402 or encrypted payload 404.

An observed retransmission 532 may also be used to update routing state. For example, the LoRa relay node 110 may observe another LoRa relay node 110 retransmitting an encrypted LoRa packet 116 having a matching packet sequence value 412, source-module identifier field 408, destination-gateway identifier field 410, or other relay-readable value. The observed retransmission 532 may indicate that a route through the observed relay node is available or that duplicate retransmission should be suppressed.

A route-table update 534 may be performed based on an acknowledgement frame 530, observed retransmission 532, signal-quality value 512, hop-path value 514, successful unicast transmission, successful broadcast transmission, gateway response, route-discovery result, or other relay-readable event. The route-table update 534 may add, refresh, rank, or modify a next-hop node entry 524 for a destination. A route-table removal 536 may be performed when acknowledgement is not detected, a unicast transmission fails, a next-hop node entry 524 expires, signal quality falls below a threshold, a hop path becomes invalid, or a route is otherwise determined to be unavailable.

Duplicate-packet suppression logic 538 may determine whether the encrypted LoRa packet 116 duplicates a previously received, stored, forwarded, retransmitted, or suppressed packet. The duplicate-packet suppression logic 538 may compare packet sequence value 412, source-module identifier field 408, destination-gateway identifier field 410, relay timestamp 516, packet signature 428, checksum 430, or another relay-readable value. If a duplicate packet is detected, the LoRa relay node 110 may suppress retransmission, update relay metadata 508, store duplicate-reception evidence, or forward the packet only when a route or priority condition requires forwarding.

A relay retransmitter 540 transmits or retransmits the encrypted LoRa packet 116 toward the authorized gateway 112. The relay retransmitter 540 may transmit in broadcast transmission mode 526, unicast transmission mode 528, store-and-forward mode, emergency mode, delayed mode, retry mode, low-power mode, or another relay mode. The relay retransmitter 540 transmits the encrypted LoRa packet 116 using relay-readable information from the relay header 400 and without decrypting the encrypted payload 404 or accessing the protected clinical-context metadata 402 in readable form.

In one example operation, the LoRa relay node 110 receives the encrypted LoRa packet 116 through the relay packet receiver 500. The relay-header parser 502 reads the relay header 400 and the relay forwarding decision logic 504 determines that forwarding is permitted. The hop-count or time-to-live update logic 506 updates the hop count or time-to-live value 414, the relay metadata 508 is appended or stored, and the encrypted LoRa packet 116 is placed in the retransmission queue 518. If the route table 522 contains a valid next-hop node entry 524, the relay retransmitter 540 transmits the encrypted LoRa packet 116 using unicast transmission mode 528. If the route table 522 does not contain a valid next-hop node entry 524, the relay retransmitter 540 transmits the encrypted LoRa packet 116 using broadcast transmission mode 526.

In another example operation, the LoRa relay node 110 transmits the encrypted LoRa packet 116 using unicast transmission mode 528 but does not receive an acknowledgement frame 530 or observe a retransmission 532 within an expected time. The LoRa relay node 110 performs route-table removal 536, removes or marks stale the next-hop node entry 524, and retransmits the encrypted LoRa packet 116 using broadcast transmission mode 526. If a later acknowledgement frame 530 or observed retransmission 532 is detected, the LoRa relay node 110 performs a route-table update 534.

In another example operation, the LoRa relay node 110 receives an encrypted LoRa packet 116 and determines that another relay node has already forwarded a packet having the same packet sequence value 412 and destination-gateway identifier field 410. The duplicate-packet suppression logic 538 suppresses retransmission by the LoRa relay node 110, thereby reducing packet duplication, congestion, power use, and collision risk while preserving the encrypted payload 404 and protected clinical-context metadata 402.

The relay-node forwarding workflow of FIG. 5 therefore supports private LoRa communication, LoRa mesh communication, adaptive routing, broadcast fallback, unicast routing, route learning, acknowledgement-based path correction, duplicate suppression, store-and-forward handling, and packet prioritization without requiring the LoRa relay nodes 110 to read protected clinical-context metadata 402 or medical-device payload data. The authorized gateway 112, rather than the LoRa relay nodes 110, remains responsible for validating clinical context and controlling downstream clinical release 138.

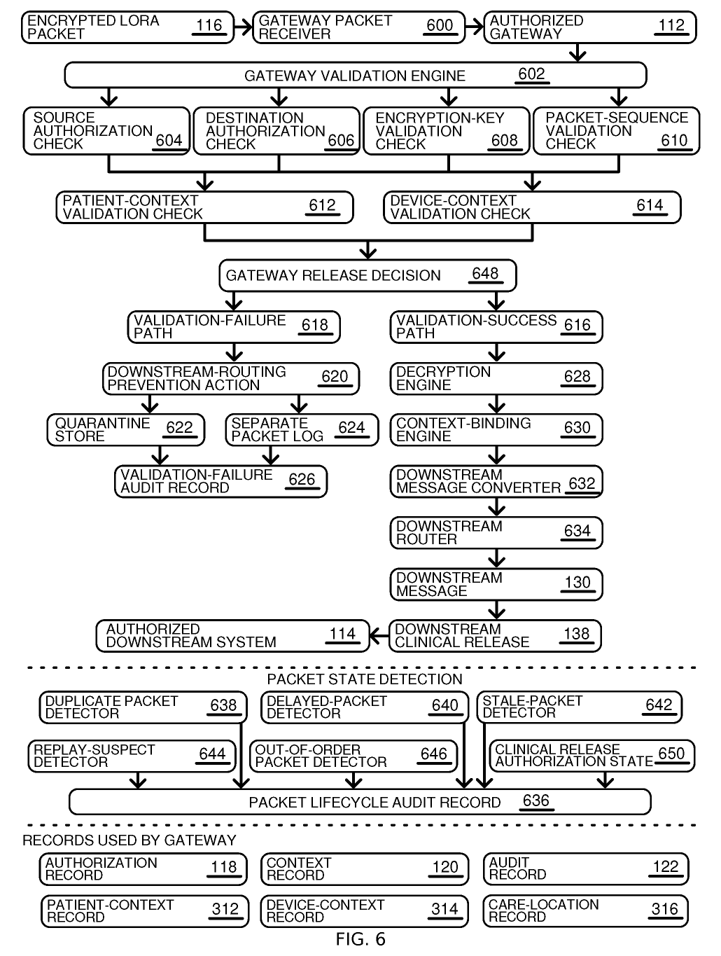

FIG. 6 — Authorized Gateway Validation, Quarantine, Conversion, Routing, and Audit

FIG. 6 illustrates an example authorized-gateway workflow in which an authorized gateway 112 receives an encrypted LoRa packet 116, validates packet information and clinical context before downstream clinical release 138, prevents downstream routing when validation is unsuccessful, and decrypts, binds, converts, routes, and audits packet data when validation is successful.

The authorized gateway 112 may be implemented as a hospital gateway, room gateway, bed gateway, wall gateway, vehicle gateway, home gateway, cloud gateway, medical device data system gateway, medical data governance gateway, or another gateway configured to receive LoRa-originated medical-device data. The authorized gateway 112 may include or communicate with gateway processing and memory resources configured to store or access one or more authorization records 118, one or more context records 120, and one or more audit records 122.

A gateway packet receiver 600 receives the encrypted LoRa packet 116 from a LoRa communication module 102 or from one or more LoRa relay nodes 110 over a LoRa communication path 134. The encrypted LoRa packet 116 may include a relay header 400, protected clinical-context metadata 402, and an encrypted payload 404. In some embodiments, the gateway packet receiver 600 receives the encrypted LoRa packet 116 directly from the LoRa communication module 102. In other embodiments, the gateway packet receiver 600 receives the encrypted LoRa packet 116 after the encrypted LoRa packet 116 has been forwarded, stored, retransmitted, or relayed by one or more LoRa relay nodes 110.

A gateway validation engine 602 evaluates the encrypted LoRa packet 116 before downstream clinical release 138. In some embodiments, the gateway validation engine 602 performs one or more validation operations before decrypting the encrypted payload 404. In other embodiments, limited gateway-readable protected metadata may be accessed or decrypted first, while patient-identifying, device-identifying, or clinical payload information remains protected until validation is complete. The gateway validation engine 602 may validate packet information against one or more authorization records 118, patient-context records 312, device-context records 314, care-location records 316, context records 120, gateway records, downstream-destination records, or governance policies.

A source authorization check 604 determines whether the encrypted LoRa packet 116 corresponds to an authorized source. The source authorization check 604 may use the source-module identifier field 408, source-module identifier 304, device identifier 302, packet signature 428, encryption-key identifier field 418, patient-context reference field 420, device-context reference field 422, or other packet information. The source authorization check 604 may verify that the LoRa communication module 102, host medical device 104, patient-associated medical device 106, or other source is permitted to transmit data to the authorized gateway 112.

A destination authorization check 606 determines whether the encrypted LoRa packet 116 is addressed to, permitted for, or otherwise associated with the authorized gateway 112 or an authorized downstream system 114. The destination authorization check 606 may use the destination-gateway identifier field 410, gateway identifier 324, routing information 406, authorization record 118, downstream destination, or gateway association. The destination authorization check 606 may prevent packets addressed to another gateway, gateway group, facility, department, incident, care area, or downstream destination from being clinically released by the authorized gateway 112.

An encryption-key validation check 608 determines whether an encryption-key identifier, key state, packet signature, certificate, token, or cryptographic association is valid. The encryption-key validation check 608 may use the encryption-key identifier field 418, packet signature 428, protected clinical-context metadata 402, authorization record 118, context record 120, or a secure key store. The encryption-key validation check 608 may determine whether a key is authorized, active, expired, revoked, mismatched, rotated, session-specific, patient-specific, device-specific, gateway-specific, or otherwise valid for the encrypted LoRa packet 116.

A packet-sequence validation check 610 determines whether the packet sequence value 412 is expected, duplicated, delayed, stale, replay-suspect, missing, out of order, or otherwise inconsistent. The packet-sequence validation check 610 may compare the packet sequence value 412 with previously received packet sequence values from the same source-module identifier 304, device identifier 302, patient-context record 312, device-context record 314, or session identifier 322. The packet-sequence validation check 610 may be used for duplicate suppression, delayed packet handling, replay detection, missing sequence detection, reconciliation, or audit.

A patient-context validation check 612 determines whether the encrypted LoRa packet 116 corresponds to a valid patient context. The patient-context validation check 612 may use the patient-context reference field 420, patient-context record 312, patient identifier 300, patient-device association 326, room identifier 318, bed identifier 320, care-location record 316, session identifier 322, authorization record 118, or association confirmation input 334. The patient-context validation check 612 may prevent downstream clinical release 138 when a packet is not associated with the correct patient, encounter, room, bed, session, or care event.

A device-context validation check 614 determines whether the encrypted LoRa packet 116 corresponds to a valid device context. The device-context validation check 614 may use the device-context reference field 422, device-context record 314, device identifier 302, source-module identifier 304, host medical device 104, patient-associated medical device 106, device-location association 328, gateway identifier 324, room identifier 318, bed identifier 320, device state, alarm state, powered state, maintenance state, or calibration state. The device-context validation check 614 may prevent downstream clinical release 138 when a packet is not associated with the correct device, module, location, gateway, or assigned patient.

When the gateway validation engine 602 determines that the encrypted LoRa packet 116 satisfies applicable validation checks, the workflow follows a validation-success path 616. When the gateway validation engine 602 determines that one or more validation checks are unsuccessful, missing, expired, mismatched, or unauthorized, the workflow follows a validation-failure path 618.

Along the validation-failure path 618, the authorized gateway 112 performs a downstream-routing prevention action 620. The downstream-routing prevention action 620 prevents the encrypted LoRa packet 116, encrypted payload 404, or decrypted payload data from being routed, displayed, stored as clinically valid data, or otherwise released to an authorized downstream system 114. In some embodiments, the downstream-routing prevention action 620 prevents downstream clinical release 138 until a user confirmation, context correction, re-pairing operation, association update, governance action, or later validation occurs.

The authorized gateway 112 may store the encrypted LoRa packet 116 in a quarantine store 622. The quarantine store 622 may retain packets that fail validation because of an invalid source, unauthorized gateway, invalid encryption key, failed packet signature, duplicate packet sequence value, patient-device mismatch, room mismatch, bed mismatch, expired context association, unauthorized downstream destination, replay-suspect state, stale timestamp, or other validation problem. The quarantine store 622 allows the packet to be reviewed, corrected, reconciled, revalidated, used as evidence of attempted transmission, or retained for audit purposes without being released clinically.

The authorized gateway 112 may also create a separate packet log 624. The separate packet log 624 may record information about a rejected, quarantined, delayed, suppressed, duplicate, or failed packet without storing the packet as clinically valid medical data. The separate packet log 624 may include one or more of a source-module identifier 304, device identifier 302, destination-gateway identifier field 410, packet sequence value 412, timestamp, validation result, failure reason, gateway identifier 324, context reference, or routing action.

A validation-failure audit record 626 may be generated when validation is unsuccessful. The validation-failure audit record 626 may identify the encrypted LoRa packet 116, source-module identifier 304, device identifier 302, authorized gateway 112, authorization record 118, context record 120, validation failure, validation-failure path 618, downstream-routing prevention action 620, quarantine store 622, separate packet log 624, and timestamp. The validation-failure audit record 626 may permit later reconstruction of why downstream clinical release 138 was prevented.

Along the validation-success path 616, a decryption engine 628 decrypts the encrypted payload 404. The decryption engine 628 may use an encryption key, session key, device key, gateway key, patient-context key, module key, rotating key, key identifier, certificate, or other cryptographic material validated by the encryption-key validation check 608. In some embodiments, the decryption engine 628 decrypts the encrypted payload 404 only after the gateway validation engine 602 determines that communication authorization and clinical context are valid.

A context-binding engine 630 binds decrypted payload data to the applicable context record 120. The context-binding engine 630 may bind the decrypted payload data to a patient-context record 312, device-context record 314, care-location record 316, session identifier 322, patient-device association 326, device-location association 328, authorization record 118, room identifier 318, bed identifier 320, gateway identifier 324, or downstream destination. The context-binding engine 630 may also enrich the decrypted payload data with validated context information before downstream routing.

A downstream message converter 632 converts the context-bound decrypted payload data into a downstream message 130. The downstream message 130 may be formatted as an HL7 message, FHIR resource, JSON object, XML object, MQTT message, HTTPS message, TCP/IP message, UDP message, proprietary medical-device data message, electronic medical record message, medical device data system message, medical data governance message, browser-renderable message, or another downstream-compatible message format. The downstream message converter 632 may normalize values, add timestamps, apply units, map fields, add context references, attach device identifiers, include alarm states, preserve original event times, or otherwise transform the data for downstream use.

A downstream router 634 transmits, publishes, stores, or otherwise makes the downstream message 130 available to an authorized downstream system 114. The authorized downstream system 114 may include an electronic medical record system 124, medical device data system 126, medical data governance system 128, browser interface, hospital operations system, transport dashboard, clinical command system, cloud system, or other authorized system. The downstream router 634 may select a downstream destination based on the context record 120, authorization record 118, patient-context record 312, device-context record 314, care-location record 316, packet priority field 416, downstream-destination rule, gateway policy, or governance policy.

A packet lifecycle audit record 636 may be generated or updated along the validation-success path 616. The packet lifecycle audit record 636 may link the encrypted LoRa packet 116, source-module identifier 304, device identifier 302, LoRa communication module 102, host medical device 104, patient-associated medical device 106, authorized gateway 112, authorization record 118, context record 120, patient-context record 312, device-context record 314, care-location record 316, validation-success path 616, decryption engine 628, context-binding engine 630, downstream message converter 632, downstream router 634, downstream message 130, authorized downstream system 114, downstream clinical release 138, and timestamp.

The authorized gateway 112 may include a duplicate packet detector 638. The duplicate packet detector 638 may compare a packet sequence value 412, source-module identifier 304, device identifier 302, packet signature 428, checksum 430, event timestamp, received timestamp, or other packet information to determine whether a duplicate packet has been received. When a duplicate is detected, the authorized gateway 112 may suppress duplicate downstream routing, store a duplicate-packet record, update the packet lifecycle audit record 636, or use the duplicate as evidence of route redundancy, signal quality, or gateway coverage.

The authorized gateway 112 may include a delayed-packet detector 640, stale-packet detector 642, replay-suspect detector 644, and out-of-order packet detector 646. The delayed-packet detector 640 may determine whether a packet arrived after an expected time window. The stale-packet detector 642 may determine whether the packet is too old for real-time clinical release. The replay-suspect detector 644 may determine whether a packet may have been improperly retransmitted or reused. The out-of-order packet detector 646 may determine whether packet sequence values were received in an unexpected order. These detectors may cause the authorized gateway 112 to add a delay indicator, suppress real-time alerting, request confirmation, route data with a warning, quarantine the packet, reconcile the packet with prior data, or update the audit record 122.

A gateway release decision 648 determines whether the decrypted payload data, downstream message 130, or context-bound data may be released to an authorized downstream system 114. The gateway release decision 648 may depend on the gateway validation engine 602, validation-success path 616, validation-failure path 618, duplicate packet detector 638, delayed-packet detector 640, stale-packet detector 642, replay-suspect detector 644, out-of-order packet detector 646, authorization record 118, context record 120, governance policy, or user confirmation. A clinical release authorization state 650 may represent whether the packet or downstream message is authorized, blocked, quarantined, delayed, duplicate-suppressed, warning-marked, pending confirmation, or released.

In operation, the authorized gateway 112 receives the encrypted LoRa packet 116 through the gateway packet receiver 600. The gateway validation engine 602 validates communication authorization, cryptographic association, packet sequence, patient context, and device context. If validation fails, the authorized gateway 112 follows the validation-failure path 618, performs the downstream-routing prevention action 620, optionally stores the packet in the quarantine store 622, creates a separate packet log 624, and generates the validation-failure audit record 626. If validation succeeds, the authorized gateway 112 follows the validation-success path 616, decrypts the encrypted payload 404 using the decryption engine 628, binds the decrypted payload data using the context-binding engine 630, converts the data using the downstream message converter 632, routes the downstream message 130 using the downstream router 634, and generates or updates the packet lifecycle audit record 636.

The workflow of FIG. 6 therefore provides an authorized-gateway control point between LoRa-originated packet transmission and downstream clinical release 138. The authorized gateway 112 does not merely receive and forward LoRa data. Rather, the authorized gateway 112 determines whether the encrypted LoRa packet 116 is associated with an authorized source, authorized destination, valid patient context, valid device context, valid communication path, and valid downstream destination before the data may become clinically available in an authorized downstream system 114.

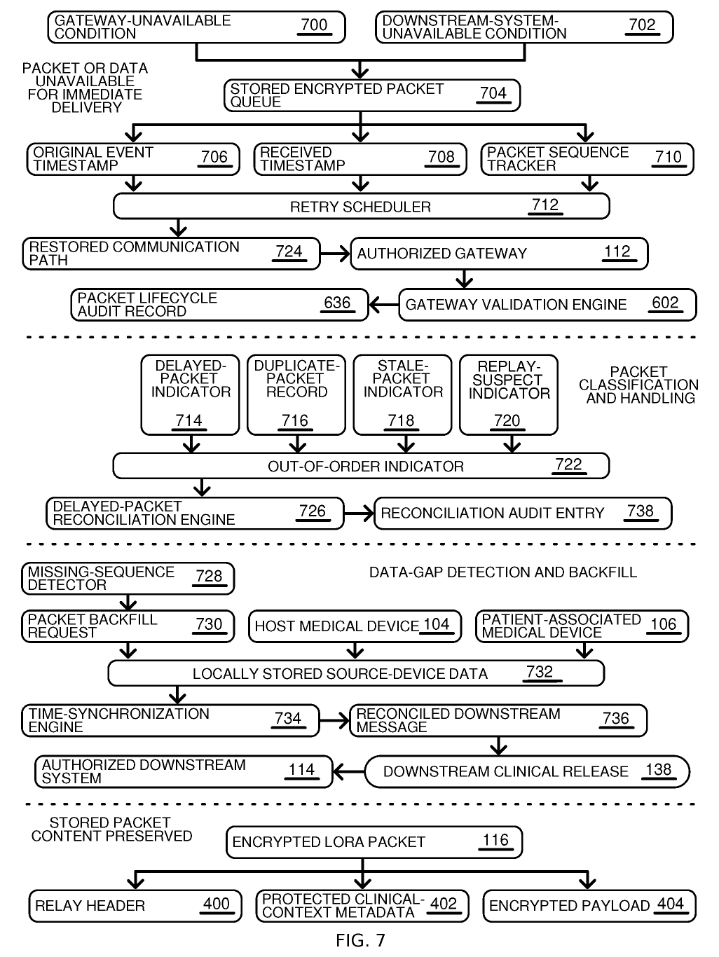

FIG. 7 — Store-and-Forward and Delayed Packet Reconciliation

FIG. 7 illustrates an example store-and-forward and delayed packet reconciliation workflow in which an encrypted LoRa packet 116 may be stored, sequenced, retried, forwarded, reconciled, suppressed, flagged, backfilled, time-synchronized, or audited when a LoRa communication path 134, authorized gateway 112, authorized downstream system 114, or other communication component is temporarily unavailable, congested, degraded, or unable to receive data.

The workflow of FIG. 7 may be performed by a LoRa communication module 102, one or more LoRa relay nodes 110, an authorized gateway 112, a medical device data system 126, a medical data governance system 128, or a distributed combination thereof. In some embodiments, the workflow supports packet continuity where encrypted LoRa packets 116 have already been generated but cannot immediately reach the authorized gateway 112. In other embodiments, the workflow supports clinical data continuity where source data 108 remains stored in or near a host medical device 104 or patient-associated medical device 106 and must be retrieved later to fill a missing data segment.

A gateway-unavailable condition 700 may occur when the authorized gateway 112 is temporarily unreachable, powered off, disconnected, overloaded, out of LoRa range, outside a permitted gateway association, unable to validate packets, or otherwise unavailable to receive or process the encrypted LoRa packet 116. The gateway-unavailable condition 700 may be detected by the LoRa communication module 102, LoRa relay node 110, another relay node, an anchor node, a store-and-forward node, or the authorized gateway 112 itself.

A downstream-system-unavailable condition 702 may occur when the authorized downstream system 114, electronic medical record system 124, medical device data system 126, medical data governance system 128, hospital operations system, cloud system, browser system, or other downstream destination is temporarily unreachable, offline, congested, unauthorized, or unable to receive a downstream message 130. In some embodiments, the authorized gateway 112 may still receive and validate the encrypted LoRa packet 116 but may delay downstream routing until the downstream-system-unavailable condition 702 is resolved.

A stored encrypted packet queue 704 stores encrypted LoRa packets 116 pending forwarding, validation, routing, reconciliation, or downstream release. The stored encrypted packet queue 704 may be maintained in local buffer 236, store-and-forward memory 520, gateway memory, downstream system memory, medical data governance memory, or another storage component. The stored encrypted packet queue 704 stores the encrypted LoRa packet 116 without requiring relay nodes or intermediate storage components to decrypt the encrypted payload 404 or access protected clinical-context metadata 402 in readable form.

An original event timestamp 706 identifies the time at which source data 108 was generated, measured, received from the host medical device 104, received from the patient-associated medical device 106, or otherwise associated with a clinical event. The original event timestamp 706 may be included in the protected clinical-context metadata 402, encrypted payload 404, context record 120, packet lifecycle audit record 636, local buffer 236, stored encrypted packet queue 704, or another record. The original event timestamp 706 may be preserved even when the encrypted LoRa packet 116 is delayed, stored, retransmitted, or forwarded after the original event.

A received timestamp 708 identifies the time at which the encrypted LoRa packet 116 is received by a LoRa relay node 110, authorized gateway 112, gateway packet receiver 600, medical device data system 126, medical data governance system 128, or authorized downstream system 114. The received timestamp 708 may be compared with the original event timestamp 706 to determine a delay interval, delayed-packet state, stale-packet state, replay-suspect state, or reconciliation requirement.

A packet sequence tracker 710 tracks packet sequence values 412 associated with one or more encrypted LoRa packets 116. The packet sequence tracker 710 may be maintained by the LoRa communication module 102, LoRa relay node 110, authorized gateway 112, medical device data system 126, medical data governance system 128, or distributed combination thereof. The packet sequence tracker 710 may detect missing sequence values, duplicate sequence values, out-of-order packet arrival, retransmitted packets, delayed packets, or gaps in a sequence of packets associated with a source-module identifier 304, device identifier 302, patient-context record 312, device-context record 314, session identifier 322, or other source.

A retry scheduler 712 controls retransmission timing for encrypted LoRa packets 116 stored in the stored encrypted packet queue 704. The retry scheduler 712 may adjust a retry interval, retry count, transmission priority, hop count, time-to-live value, broadcast mode, unicast mode, packet priority field 416, or route-selection parameter based on gateway availability, downstream availability, packet urgency, alarm state, clinical context, signal quality, route-table state, or governance policy. In some embodiments, emergency, alarm, or high-priority packets may be retried more frequently or transmitted through multiple relay paths.

A delayed-packet indicator 714 may be assigned when an encrypted LoRa packet 116 is received later than expected or after a threshold delay. The delayed-packet indicator 714 may be stored in the packet lifecycle audit record 636, audit record 122, downstream message 130, context record 120, or delayed-packet reconciliation record. The delayed-packet indicator 714 may cause the authorized gateway 112 to route the downstream message 130 with a delay warning, suppress real-time alerting, preserve the original event timestamp 706, request user confirmation, or route the packet for reconciliation instead of immediate clinical display.

A duplicate-packet record 716 may be generated when a duplicate packet is detected. The duplicate-packet record 716 may be created by the duplicate packet detector 638, packet sequence tracker 710, authorized gateway 112, medical data governance system 128, or another component. The duplicate-packet record 716 may include a source-module identifier 304, packet sequence value 412, packet signature 428, original event timestamp 706, received timestamp 708, gateway identifier 324, relay metadata 508, signal-quality value 512, hop-path value 514, or duplicate-suppression action. In some embodiments, duplicate packet instances may be retained as evidence of route redundancy or coverage but suppressed from duplicate downstream clinical routing.

A stale-packet indicator 718 may be assigned when the encrypted LoRa packet 116 or source data 108 is older than a permitted clinical release threshold. A replay-suspect indicator 720 may be assigned when the encrypted LoRa packet 116 appears to be improperly repeated, reused, inconsistent with expected sequence values, inconsistent with timestamps, or otherwise suspicious. An out-of-order indicator 722 may be assigned when encrypted LoRa packets 116 are received in an order different from their packet sequence values 412 or original event timestamps 706. These indicators may be used by the authorized gateway 112, gateway validation engine 602, delayed-packet detector 640, stale-packet detector 642, replay-suspect detector 644, out-of-order packet detector 646, or medical data governance system 128 to determine whether to route, suppress, quarantine, reconcile, flag, or audit the packet.

A restored communication path 724 represents restoration of connectivity between the LoRa communication module 102, LoRa relay node 110, authorized gateway 112, authorized downstream system 114, medical device data system 126, medical data governance system 128, or another communication component. Once the restored communication path 724 is available, encrypted LoRa packets 116 in the stored encrypted packet queue 704 may be forwarded, validated, decrypted, bound to context, converted into downstream messages 130, routed, reconciled, or audited.

A delayed-packet reconciliation engine 726 reconciles delayed, stored, retransmitted, duplicate, stale, replay-suspect, out-of-order, or backfilled data with previously received data. The delayed-packet reconciliation engine 726 may compare the original event timestamp 706, received timestamp 708, packet sequence value 412, packet sequence tracker 710, context record 120, patient-context record 312, device-context record 314, session identifier 322, downstream message 130, and packet lifecycle audit record 636. The delayed-packet reconciliation engine 726 may insert delayed data into a time-ordered clinical data stream, mark the data as delayed, suppress duplicate data, request confirmation, quarantine suspicious data, or generate a reconciliation audit entry 738.

A missing-sequence detector 728 determines whether one or more expected packet sequence values 412 or time segments are missing. The missing-sequence detector 728 may detect a gap in encrypted LoRa packets 116, a gap in source data 108, a gap in downstream messages 130, or a gap in data associated with a patient-context record 312, device-context record 314, session identifier 322, or host medical device 104. In some embodiments, the missing-sequence detector 728 may identify that packet transmission was interrupted during patient movement, relay-node loss, gateway loss, network congestion, power interruption, or communication-path unavailability.

A packet backfill request 730 may be generated when a missing packet sequence, missing time segment, or missing source-data segment is detected. The packet backfill request 730 may be transmitted from the authorized gateway 112, medical device data system 126, medical data governance system 128, or another authorized system to the LoRa communication module 102, host medical device 104, patient-associated medical device 106, or another data source. The packet backfill request 730 may request locally stored source-device data 732 corresponding to a missing sequence value, time interval, original event timestamp range, device session, patient-context record 312, device-context record 314, or other missing segment.

Locally stored source-device data 732 may include data retained in the host medical device 104, patient-associated medical device 106, LoRa communication module 102, local buffer 236, memory 204, wearable device memory, phone memory, sensor memory, monitor memory, or another local storage element. The locally stored source-device data 732 may include medical-device data, physiological data, alarm data, powered-state data, battery data, device-use data, device-identity data, waveform data, vital-sign data, or other source data 108. In some embodiments, the locally stored source-device data 732 is transmitted after reconnection to fill a missing data segment.

A time-synchronization engine 734 aligns delayed, stored, retransmitted, or backfilled data with previously received data. The time-synchronization engine 734 may use original event timestamps 706, received timestamps 708, packet sequence values 412, device clocks, gateway clocks, session identifiers 322, care-location records 316, context records 120, or medical data governance time references. The time-synchronization engine 734 may insert locally stored source-device data 732 into a chronological data stream, label delayed data, preserve original event time, distinguish original measurement time from received time, or reconcile overlapping or duplicate data.

A reconciled downstream message 736 may be generated after delayed-packet reconciliation, backfill, duplicate suppression, time synchronization, or missing-segment recovery. The reconciled downstream message 736 may include delayed data, backfilled data, corrected data, time-synchronized data, duplicate-suppressed data, or gap-repaired data. The reconciled downstream message 736 may be sent to the authorized downstream system 114, electronic medical record system 124, medical device data system 126, medical data governance system 128, or another authorized destination.

A reconciliation audit entry 738 may be generated to record how a delayed, stored, duplicate, stale, replay-suspect, out-of-order, or backfilled packet was handled. The reconciliation audit entry 738 may identify the encrypted LoRa packet 116, source-module identifier 304, device identifier 302, patient-context record 312, device-context record 314, original event timestamp 706, received timestamp 708, packet sequence value 412, delayed-packet indicator 714, duplicate-packet record 716, stale-packet indicator 718, replay-suspect indicator 720, out-of-order indicator 722, packet backfill request 730, locally stored source-device data 732, time-synchronization action, reconciled downstream message 736, and final routing or suppression action.

In one example operation, the LoRa communication module 102 generates an encrypted LoRa packet 116, but a gateway-unavailable condition 700 prevents immediate delivery to the authorized gateway 112. The encrypted LoRa packet 116 is placed in the stored encrypted packet queue 704, associated with an original event timestamp 706 and packet sequence value 412, and managed by the retry scheduler 712. When the restored communication path 724 becomes available, the encrypted LoRa packet 116 is forwarded to the authorized gateway 112, which validates, decrypts, binds, converts, routes, and audits the packet. If the packet is delayed, the delayed-packet indicator 714 is added and the delayed-packet reconciliation engine 726 reconciles the data with previously received data.

In another example operation, the authorized gateway 112 or medical data governance system 128 detects a missing packet sequence value using the missing-sequence detector 728. The system generates a packet backfill request 730 to retrieve locally stored source-device data 732 from the host medical device 104, patient-associated medical device 106, or LoRa communication module 102. The locally stored source-device data 732 is transmitted after reconnection, time-synchronized by the time-synchronization engine 734, and incorporated into a reconciled downstream message 736. A reconciliation audit entry 738 records the backfill and time-synchronization action.

In another example operation, multiple LoRa relay nodes 110 forward duplicate instances of the same encrypted LoRa packet 116 through different relay paths. The duplicate packet detector 638 or packet sequence tracker 710 detects the duplicate based on the packet sequence value 412, source-module identifier 304, packet signature 428, or original event timestamp 706. The system stores a duplicate-packet record 716, suppresses duplicate downstream routing, and may retain duplicate reception information for signal-quality analysis, route analysis, gateway-coverage analysis, or audit.

The workflow of FIG. 7 therefore supports continuity of medical-device data during intermittent, delayed, interrupted, or redundant LoRa communication. By preserving packet sequence values 412, original event timestamps 706, received timestamps 708, context records 120, and audit records 122, the system can distinguish real-time data, delayed data, duplicate data, stale data, replay-suspect data, out-of-order data, and backfilled data before downstream clinical release 138.

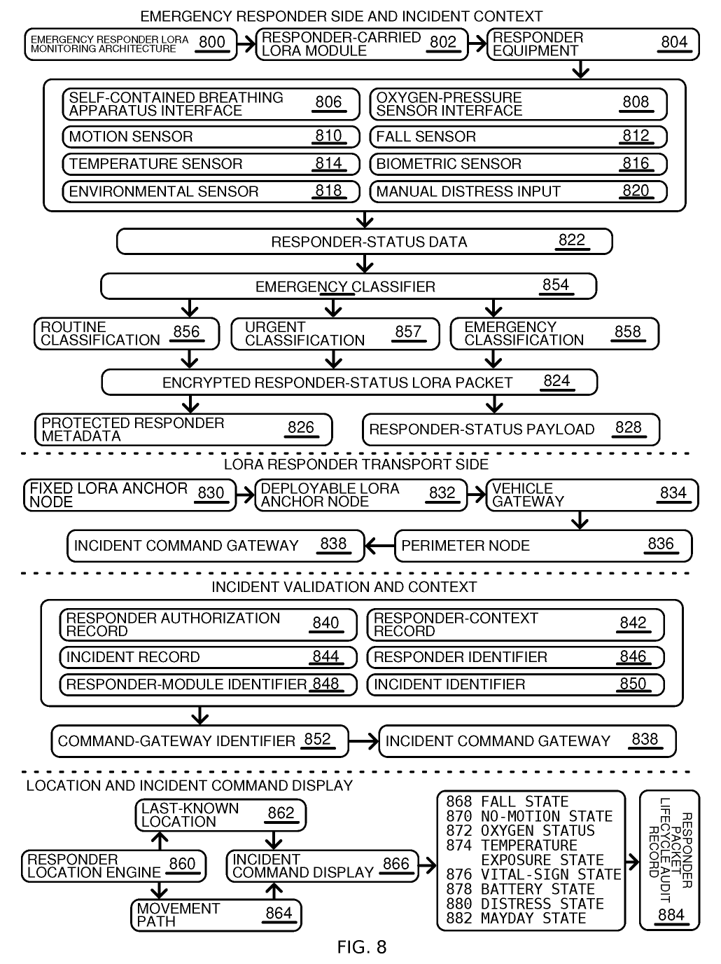

FIG. 8 — Emergency Responder LoRa Monitoring and Incident Command Display

FIG. 8 illustrates an example emergency responder LoRa monitoring architecture 800 configured to obtain responder-status data 822 from responder equipment 804, generate an encrypted responder-status LoRa packet 824, transmit the encrypted responder-status LoRa packet 824 through one or more LoRa anchor, relay, mesh, vehicle, or perimeter nodes, and make validated responder-status information available to an incident command gateway 838 and an incident command display 866.

The emergency responder LoRa monitoring architecture 800 may be used in connection with a firefighter, emergency medical technician, police officer, hazmat worker, search-and-rescue worker, military medic, industrial safety worker, disaster-response worker, confined-space worker, or other emergency responder. The architecture 800 may be deployed in a building, fireground, industrial site, tunnel, mine, ship, aircraft, ambulance, field hospital, disaster-response site, remote-care site, military environment, hazardous environment, or other incident environment where conventional cellular, Wi-Fi, wired, or short-range communication may be unavailable, degraded, unsafe, or unreliable.

A responder-carried LoRa module 802 is attached to, embedded in, clipped to, plugged into, integrated with, or otherwise associated with responder equipment 804. The responder equipment 804 may include turnout gear, helmet, belt, harness, backpack strap, self-contained breathing apparatus, oxygen apparatus, responder bracelet, medical patch, wearable sensor, handheld device, vehicle equipment, rescue equipment, personal protective equipment, or other equipment carried by or associated with a responder.

The responder-carried LoRa module 802 may include a LoRa transceiver, processor, memory, encryption logic, power source, local communication interface, antenna, sensor interface, module firmware, local buffer, and packet generation logic, similar to corresponding components of the LoRa communication module 102 described with respect to earlier figures. In some embodiments, the responder-carried LoRa module 802 is configured for responder safety, hazardous-environment operation, fire resistance, impact resistance, waterproofing, intrinsic safety, replaceable-battery operation, low-power operation, emergency transmission, or store-and-forward operation.

The responder-carried LoRa module 802 may receive responder-status data 822 through one or more responder-status interfaces. The responder-status interfaces may include a self-contained breathing apparatus interface 806, oxygen-pressure sensor interface 808, motion sensor 810, fall sensor 812, temperature sensor 814, biometric sensor 816, environmental sensor 818, manual distress input 820, helmet sensor, turnout-gear sensor, responder equipment interface, or other responder-associated input.

The self-contained breathing apparatus interface 806 may receive data from a self-contained breathing apparatus, oxygen apparatus, breathing-air tank, oxygen tank, oxygen regulator, air-pressure monitor, breathing apparatus controller, or other respiratory support equipment. The oxygen-pressure sensor interface 808 may receive oxygen pressure, remaining air time, oxygen flow, oxygen concentration, oxygen alarm state, valve state, or related oxygen-status information. The motion sensor 810 may include an accelerometer, gyroscope, inertial measurement unit, step detector, vibration sensor, orientation sensor, or other movement sensor. The fall sensor 812 may detect a fall event, impact event, posture change, tilt condition, collapse condition, or no-motion condition. The temperature sensor 814 may measure ambient temperature, gear temperature, skin temperature, heat exposure, thermal stress, or another temperature condition. The biometric sensor 816 may measure heart rate, respiratory rate, oxygen saturation, temperature, exertion, stress, or other physiological data. The environmental sensor 818 may measure smoke, gas, toxic exposure, humidity, pressure, heat, atmospheric condition, or another environmental state. The manual distress input 820 may be a button, switch, gesture input, voice input, touch input, pull cord, panic input, Mayday input, or other user-actuated distress mechanism.

The responder-status data 822 may include responder identity data, responder-status data, location data, motion data, no-motion data, fall data, distress data, Mayday data, vital-sign data, oxygen status data, temperature exposure data, battery data, equipment-status data, incident event data, environmental data, or other responder-associated information. The responder-status data 822 may be received periodically, continuously, intermittently, upon a threshold crossing, upon an alarm condition, upon a motion change, upon loss of motion, upon oxygen depletion, upon temperature exposure, upon manual activation, upon gateway loss, or according to a command, rule, policy, or responder assignment.

The responder-carried LoRa module 802 generates an encrypted responder-status LoRa packet 824. The encrypted responder-status LoRa packet 824 may include a relay-readable header, protected responder metadata 826, and a responder-status payload 828. The relay-readable header may include routing information, responder-module identifier 848, command-gateway identifier 852, packet sequence value, hop count or time-to-live value, packet priority, packet type, checksum, or other relay-readable information. The protected responder metadata 826 may include a responder-context reference, incident identifier 850, encryption-key identifier, team identifier, equipment identifier, location reference, gateway identifier, event timestamp, downstream destination identifier, routing-rule identifier, quarantine-rule identifier, audit-rule identifier, or other protected responder or incident context. The responder-status payload 828 may include encrypted responder-status data 822, including oxygen status, fall state, no-motion state, distress state, Mayday state, vital-sign state, temperature exposure state, battery state, location data, or equipment-status data.

The responder-carried LoRa module 802 may classify the responder-status data 822 before or during packet generation. An emergency classifier 854 may classify the responder-status data 822 as a routine classification 856, an urgent classification 857, or an emergency classification 858. The emergency classifier 854 may evaluate oxygen status, oxygen-pressure data, remaining air time, no-motion duration, fall state, temperature exposure, vital-sign state, manual distress input, Mayday input, battery state, gateway-loss state, environmental condition, or combinations thereof. In one example, the emergency classifier 854 classifies responder-status data 822 as emergency data when oxygen status data satisfies a low-oxygen threshold and motion data indicates a no-motion condition for a threshold time period. In another example, activation of the manual distress input 820 causes emergency classification 858 and generation of an emergency-priority encrypted responder-status LoRa packet 824.

The encrypted responder-status LoRa packet 824 may be transmitted directly to the incident command gateway 838 or through one or more fixed LoRa anchor nodes 830, deployable LoRa anchor nodes 832, vehicle gateways 834, perimeter nodes 836, LoRa relay nodes, mesh nodes, repeaters, store-and-forward nodes, or other LoRa-capable nodes. A fixed LoRa anchor node 830 may be installed in or near a building, facility, floor, stairwell, hallway, tunnel, industrial area, field site, command area, or known location. A deployable LoRa anchor node 832 may be temporarily placed by responders, incident command personnel, vehicles, drones, robots, or support teams. A vehicle gateway 834 may be located in a fire truck, ambulance, command vehicle, rescue vehicle, police vehicle, aircraft, boat, or other vehicle. A perimeter node 836 may be positioned around an incident scene, building perimeter, hazard boundary, search area, staging area, or evacuation route.

The fixed LoRa anchor nodes 830, deployable LoRa anchor nodes 832, vehicle gateways 834, perimeter nodes 836, or other relay nodes forward the encrypted responder-status LoRa packet 824 toward the incident command gateway 838 using relay-readable packet information. Such nodes may update hop count, time-to-live value, relay metadata, signal-quality values, timestamps, route-table entries, or store-and-forward state without decrypting the responder-status payload 828 and without accessing protected responder metadata 826 in readable form. Accordingly, responder-status packets may be relayed through temporary, mobile, public-safety, or non-clinical nodes while preserving responder identity, incident context, and responder-status payload confidentiality.

The incident command gateway 838 receives the encrypted responder-status LoRa packet 824 directly or through one or more intermediate LoRa nodes. The incident command gateway 838 may be located at an incident command post, vehicle, mobile command center, field hospital, dispatch center, hospital command center, cloud system, emergency operations center, or another command location. The incident command gateway 838 may include or access one or more responder authorization records 840, responder-context records 842, and incident records 844.

The responder authorization record 840 may identify a responder-carried LoRa module 802, responder identifier 846, responder-module identifier 848, team assignment, equipment assignment, incident assignment, encryption key, command-gateway authorization, gateway association, role authorization, or permitted downstream destination. The responder-context record 842 may identify a responder, responder role, team identifier, assigned equipment, responder status, last-known location, gateway association, movement history, alert state, battery state, oxygen status, or other responder context. The incident record 844 may identify an incident, incident identifier 850, building, floor, zone, command assignment, incident location, incident scene, deployment area, authorized command gateway, responder roster, team assignment, or incident-specific policy.

A responder identifier 846 may identify a responder, firefighter, medic, police officer, hazmat worker, search-and-rescue worker, military medic, industrial safety worker, or other emergency responder. The responder-module identifier 848 may identify the responder-carried LoRa module 802. The incident identifier 850 may identify the incident, event, response assignment, command operation, rescue operation, training event, or emergency deployment. The command-gateway identifier 852 may identify the incident command gateway 838 or a gateway group authorized to receive and validate the encrypted responder-status LoRa packet 824.

Before displaying or releasing responder-status information, the incident command gateway 838 validates the encrypted responder-status LoRa packet 824. The incident command gateway 838 may validate the responder-module identifier 848, responder identifier 846, incident identifier 850, command-gateway identifier 852, encryption-key identifier, packet sequence value, protected responder metadata 826, responder authorization record 840, responder-context record 842, incident record 844, gateway association, team assignment, or downstream destination. When validation is unsuccessful, the incident command gateway 838 may prevent display or downstream routing, quarantine or separately log the packet, generate a validation-failure audit record, request reassignment, require confirmation, or suppress an unauthorized packet.

When validation is successful, the incident command gateway 838 decrypts the responder-status payload 828 and binds the decrypted responder-status data to the responder-context record 842 and incident record 844 identified by the protected responder metadata 826. The incident command gateway 838 may determine, update, or display responder location, movement path, last-known location, fall state, no-motion state, oxygen status, temperature exposure, vital-sign state, battery state, distress state, Mayday state, equipment state, incident assignment, or other responder status.

A responder location engine 860 may determine or estimate a responder location using received signal strength, time-of-arrival information, time-difference-of-arrival information, anchor proximity, gateway proximity, hop path, inertial data, barometric data, floor-plan data, building map data, LIDAR data, manual confirmation, or combinations thereof. The responder location engine 860 may determine a two-dimensional location, three-dimensional location, floor level, zone, room, stairwell, hallway, route, proximity, last-known position, confidence value, or movement trend. In some embodiments, the responder location engine 860 uses signals received by multiple fixed LoRa anchor nodes 830, deployable LoRa anchor nodes 832, vehicle gateways 834, perimeter nodes 836, or other LoRa nodes.

A last-known location 862 may be stored when communication with the responder-carried LoRa module 802 is degraded, interrupted, unavailable, delayed, or lost. The last-known location 862 may include a coordinate, map point, floor, room, zone, gateway association, anchor association, timestamp, signal-quality value, confidence value, or movement state. A movement path 864 may represent prior locations, route history, entry path, exit path, search path, movement trend, or motion history associated with the responder.

An incident command display 866 may show one or more responder status conditions. The incident command display 866 may include a map, floor plan, building model, three-dimensional model, LIDAR model, incident-scene map, command dashboard, vehicle display, tablet display, mobile command interface, browser interface, or other visual interface. The incident command display 866 may present a responder location, last-known location 862, movement path 864, fall state 868, no-motion state 870, oxygen status 872, temperature exposure state 874, vital-sign state 876, battery state 878, distress state 880, Mayday state 882, incident assignment, team assignment, equipment status, or alert state.

The fall state 868 may indicate that a responder has fallen, impacted the ground, changed posture abruptly, or is in a possible collapse condition. The no-motion state 870 may indicate that motion sensor 810 data has not changed for a threshold time period. The oxygen status 872 may indicate oxygen pressure, oxygen depletion, remaining air time, oxygen flow, oxygen alarm state, or other respiratory support state. The temperature exposure state 874 may indicate ambient heat exposure, gear temperature, body temperature, heat-stress risk, or other thermal condition. The vital-sign state 876 may indicate heart rate, respiratory rate, oxygen saturation, body temperature, exertion, or other physiologic state. The battery state 878 may indicate battery level, remaining power, power fault, replacement requirement, or charging state. The distress state 880 may indicate a general emergency, unsafe condition, responder distress, manual distress input, or other alert state. The Mayday state 882 may indicate a high-priority emergency state generated by manual activation, automated classification, incident command input, or responder-status threshold crossing.

A responder packet lifecycle audit record 884 may be generated or updated by the incident command gateway 838. The responder packet lifecycle audit record 884 may link the encrypted responder-status LoRa packet 824, responder-carried LoRa module 802, responder identifier 846, responder-module identifier 848, responder-context record 842, incident record 844, incident command gateway 838, validation result, decrypted responder-status payload, displayed responder status, location determination, downstream destination, command action, alert state, and timestamp. The responder packet lifecycle audit record 884 may permit reconstruction of responder-status receipt, validation, decryption, mapping, classification, display, routing, alerting, correction, quarantine, or audit actions.

In one example operation, the responder-carried LoRa module 802 receives oxygen-pressure data from the oxygen-pressure sensor interface 808 and motion data from the motion sensor 810. The emergency classifier 854 determines that the oxygen status 872 satisfies a low-oxygen threshold and that the no-motion state 870 has persisted for a threshold time period. The responder-carried LoRa module 802 classifies the responder-status data 822 with the emergency classification 858, encrypts the responder-status payload 828, generates the encrypted responder-status LoRa packet 824, and transmits the packet through one or more deployable LoRa anchor nodes 832 toward the incident command gateway 838. After validation, the incident command gateway 838 updates the incident command display 866 to show the responder location, oxygen status 872, no-motion state 870, distress state 880, and Mayday state 882.

In another example operation, the responder-carried LoRa module 802 receives a manual distress input 820 from a responder. The manual distress input 820 causes the emergency classifier 854 to assign emergency classification 858 and generate the encrypted responder-status LoRa packet 824 with increased packet priority, increased retry behavior, modified hop count or time-to-live value, or multiple command-gateway destinations. The encrypted responder-status LoRa packet 824 is forwarded by a fixed LoRa anchor node 830, vehicle gateway 834, or perimeter node 836 toward the incident command gateway 838. The incident command gateway 838 validates the packet, decrypts the responder-status payload 828, updates the incident command display 866, and creates or updates the responder packet lifecycle audit record 884.

The emergency responder LoRa monitoring architecture 800 therefore applies the same general principles of relay-readable routing, protected context metadata, encrypted payload handling, authorized gateway validation, downstream release control, and auditability to emergency responder safety. The architecture 800 allows responder-status packets to be relayed through fixed, deployable, vehicle-mounted, or perimeter LoRa nodes while preserving responder identity, incident context, responder-status payload confidentiality, and incident command control over display and downstream routing.

1. A medical-device LoRa communication system comprising:
a LoRa communication module comprising a LoRa transceiver, a processor, memory, encryption logic, and a local communication interface, the LoRa communication module being attached to, embedded in, plugged into, or otherwise coupled to a host medical device or patient-associated medical device;
wherein the memory stores or receives a source-module identifier, a device identifier for the host medical device or patient-associated medical device, a patient-context reference or device-context reference, an encryption-key identifier, a destination-gateway identifier, and a packet sequence counter;
wherein the local communication interface is configured to obtain medical-device data, physiological data, alarm data, powered-state data, battery data, device-use data, or device-identity data from the host medical device or patient-associated medical device through a wired medical-device interface, serial interface, USB interface, Bluetooth interface, Bluetooth Low Energy interface, NFC-assisted pairing interface, or sensor interface;
wherein the processor is configured to generate an encrypted LoRa packet by assigning a packet sequence value using the packet sequence counter, selecting an encryption key associated with the encryption-key identifier, encrypting at least a payload comprising the obtained medical-device data, physiological data, alarm data, powered-state data, battery data, device-use data, or device-identity data to generate an encrypted payload forming at least part of a relay-unreadable packet portion, generating a relay header forming at least part of a relay-readable packet portion readable by one or more LoRa relay nodes, the relay header comprising at least the source-module identifier, destination-gateway identifier, packet sequence value, hop count or time-to-live value, and packet priority, and generating protected clinical-context metadata forming at least part of the relay-unreadable packet portion, the protected clinical-context metadata being encrypted, signed, tokenized, pseudonymized, wrapped for gateway access, gateway-resolvable, or gateway-readable only, such that the protected clinical-context metadata is unavailable in readable form to the one or more LoRa relay nodes and accessible in readable or resolvable form by an authorized gateway, the protected clinical-context metadata comprising at least the patient-context reference or device-context reference and the encryption-key identifier;
wherein the encrypted LoRa packet comprises the relay-readable packet portion and the relay-unreadable packet portion, wherein the relay-readable packet portion comprises routing information usable by the one or more LoRa relay nodes to forward the encrypted LoRa packet toward an authorized gateway, and wherein the relay-unreadable packet portion comprises at least the protected clinical-context metadata and the encrypted payload;
one or more LoRa relay nodes configured to receive the encrypted LoRa packet and forward the encrypted LoRa packet toward an authorized gateway based on the relay-readable packet portion by reading the relay header, determining whether the hop count or time-to-live value permits forwarding, updating the hop count or time-to-live value, appending relay metadata to the relay-readable packet portion or to a relay-metadata field, and retransmitting the encrypted LoRa packet while preserving the relay-unreadable packet portion without decryption and without requiring readable access to the protected clinical-context metadata or the encrypted payload;
the authorized gateway comprising a gateway processor and gateway memory storing one or more authorization records and one or more context records, the authorized gateway being identified by or corresponding to the destination-gateway identifier;
wherein the authorized gateway is configured to, before decrypting the encrypted payload, receive the encrypted LoRa packet, access or resolve, in readable or resolvable form, the protected clinical-context metadata from the relay-unreadable packet portion, and validate that the source-module identifier, destination-gateway identifier, encryption-key identifier, packet sequence value, and patient-context reference or device-context reference correspond to at least one stored authorization record and at least one stored patient-context record or device-context record;
wherein, when the validation is unsuccessful, the authorized gateway is configured to prevent downstream routing of the encrypted LoRa packet, quarantine or separately log the encrypted LoRa packet, and generate a validation-failure audit record identifying at least the source-module identifier, destination-gateway identifier, packet sequence value, validation failure, and timestamp; and
wherein, when the validation is successful, the authorized gateway is configured to decrypt the encrypted payload, bind the decrypted payload to the stored patient-context record or device-context record identified by the protected clinical-context metadata accessed or resolved by the authorized gateway, convert the bound decrypted payload into a downstream electronic medical record message, medical device data system message, or medical data governance message, transmit the downstream message to an authorized downstream system, and generate a packet lifecycle audit record linking at least the encrypted LoRa packet, source-module identifier, device identifier, authorized gateway, patient-context record or device-context record, validation result, downstream message, downstream destination, and timestamp.

2. The system of claim 1, wherein the protected clinical-context metadata is included in the relay-unreadable packet portion, is inaccessible in readable form to the one or more LoRa relay nodes, and includes at least a session identifier, gateway identifier, encryption-key identifier, and at least one of a room identifier, bed identifier, or care-location identifier.

3. The system of claim 1, wherein each LoRa relay node is configured to modify only the relay-readable packet portion or relay metadata while preserving the relay-unreadable packet portion without decryption.

4. The system of claim 1, wherein the relay metadata appended by a LoRa relay node includes a relay-node identifier, received signal quality value, hop-path value, relay timestamp, and retry counter.

5. The system of claim 1, wherein the authorized gateway quarantines or separately logs the encrypted LoRa packet when the validation identifies at least one of:
an invalid encryption-key identifier;
an invalid source-module identifier;
an unauthorized destination-gateway identifier;
a duplicate packet sequence value;
a patient-device mismatch;
a room or bed mismatch;
an expired context association; or
an unauthorized downstream destination.

6. The system of claim 1, wherein the authorized gateway detects a duplicate packet by comparing the source-module identifier and packet sequence value with previously received packet records, suppresses duplicate downstream routing, and stores a duplicate-packet record linked to the packet lifecycle audit record.

7. The system of claim 1, wherein the authorized gateway determines that the encrypted LoRa packet is delayed, stale, replay-suspect, or out-of-order by comparing the packet sequence value with at least one of an original event timestamp, a received timestamp, or a previously received packet record, and routes the downstream message with a delay indicator or suppresses real-time alerting.

8. The system of claim 1, wherein the LoRa communication module or one of the LoRa relay nodes stores the encrypted LoRa packet when the authorized gateway is unavailable and forwards the stored encrypted LoRa packet after gateway availability is restored while preserving the packet sequence value, original event timestamp, and relay-unreadable packet portion.

9. The system of claim 1, wherein the patient-context record or device-context record links a patient identifier, device identifier, room identifier, bed identifier, gateway identifier, encryption-key identifier, and authorized downstream destination.

10. The system of claim 1, wherein the host medical device or patient-associated medical device comprises an infusion pump, ventilator, patient monitor, pulse oximeter, blood pressure monitor, glucose monitor, ECG monitor, oxygen apparatus, smart bed, smart IV pole, wearable sensor, medical patch, patient phone, or transport monitor.

11. The system of claim 1, wherein the downstream message comprises an HL7 message, FHIR resource, JSON object, XML object, MQTT message, HTTPS message, medical device data system message, electronic medical record message, or medical data governance message.

12. A method of operating a medical-device LoRa communication system, the method comprising:
obtaining, by a LoRa communication module coupled to a host medical device or patient-associated medical device, medical-device data, physiological data, alarm data, device-status data, device-identity data, or other patient-associated data from the host medical device or patient-associated medical device;
generating, by the LoRa communication module, an encrypted LoRa packet comprising a relay-readable packet portion and a relay-unreadable packet portion, the relay-readable packet portion comprising a relay header readable by one or more LoRa relay nodes, the relay-unreadable packet portion comprising protected clinical-context metadata inaccessible in readable form to the one or more LoRa relay nodes and an encrypted payload comprising at least a portion of the obtained medical-device data, physiological data, alarm data, device-status data, device-identity data, or other patient-associated data;
wherein the relay-readable packet portion comprises routing information usable by the one or more LoRa relay nodes to forward the encrypted LoRa packet toward an authorized gateway, and wherein the relay-unreadable packet portion is preserved by the one or more LoRa relay nodes without decryption;
transmitting the encrypted LoRa packet over a LoRa communication path toward an authorized gateway;
forwarding, by the one or more LoRa relay nodes, the encrypted LoRa packet toward the authorized gateway based on the relay-readable packet portion while preserving the relay-unreadable packet portion without decryption and without requiring readable access to the protected clinical-context metadata or the encrypted payload;
receiving, by the authorized gateway, the encrypted LoRa packet;
accessing or resolving, by the authorized gateway, the protected clinical-context metadata from the relay-unreadable packet portion;
validating, by the authorized gateway, packet information associated with the encrypted LoRa packet and the accessed or resolved protected clinical-context metadata against one or more authorization records and one or more patient-context records or device-context records before downstream clinical release of decrypted payload data;
when the validation is unsuccessful, preventing downstream clinical routing of the encrypted LoRa packet; and
when the validation is successful, decrypting, by the authorized gateway, the encrypted payload, associating decrypted payload data with at least one validated patient-context record or device-context record, and making the decrypted payload data available to an authorized downstream electronic medical record system, medical device data system, medical data governance system, or other authorized downstream system.

13. The method of claim 12, wherein the relay header comprises a source-module identifier, destination-gateway identifier, packet sequence value, hop count or time-to-live value, and packet priority, and wherein the protected clinical-context metadata is included in the relay-unreadable packet portion and comprises a patient-context reference or device-context reference and an encryption-key identifier accessible or resolvable by the authorized gateway.

14. The method of claim 12, wherein forwarding the encrypted LoRa packet comprises, by at least one LoRa relay node, determining whether a hop count or time-to-live value permits forwarding, updating the hop count or time-to-live value in the relay-readable packet portion, appending relay metadata comprising a relay-node identifier and relay timestamp, and retransmitting the encrypted LoRa packet without modifying the relay-unreadable packet portion.

15. The method of claim 12, wherein preventing downstream clinical routing comprises quarantining or separately logging the encrypted LoRa packet and generating a validation-failure audit record identifying at least a source-module identifier, destination-gateway identifier, packet sequence value, validation failure, and timestamp.

16. The method of claim 12, further comprising detecting a missing, duplicate, delayed, stale, replay-suspect, or out-of-order packet condition using at least one of a packet sequence value, an original event timestamp, or a received timestamp, and generating a reconciled downstream message or reconciliation audit entry before downstream clinical release.

17. A LoRa communication system configured for emergency responder monitoring, the system comprising:
a responder-carried LoRa module configured to be attached to, embedded in, integrated with, or otherwise associated with responder equipment;
wherein the responder-carried LoRa module is configured to obtain responder-status data from at least one responder-status interface associated with the responder equipment, the responder-status data comprising one or more of oxygen-status data, motion data, fall data, temperature data, biometric data, environmental data, battery data, equipment-status data, manual distress data, Mayday data, or location-related data;
wherein the responder-carried LoRa module is configured to generate an encrypted responder-status LoRa packet comprising a relay-readable packet portion and a relay-unreadable packet portion, the relay-readable packet portion comprising relay-readable routing information, and the relay-unreadable packet portion comprising protected responder metadata and an encrypted responder-status payload, the protected responder metadata comprising responder-context or incident-context metadata inaccessible in readable form to one or more LoRa relay nodes and accessible or resolvable by an authorized incident command gateway, and the encrypted responder-status payload comprising at least a portion of the responder-status data;
wherein the relay-readable packet portion comprises routing information usable by the one or more LoRa relay nodes to forward the encrypted responder-status LoRa packet toward an authorized incident command gateway, and wherein the relay-unreadable packet portion is preserved by the one or more LoRa relay nodes without decryption;
one or more LoRa relay nodes comprising fixed, vehicle-mounted, deployable, perimeter, anchor, mesh, or relay nodes, the one or more LoRa relay nodes being configured to forward the encrypted responder-status LoRa packet toward an authorized incident command gateway based on the relay-readable packet portion while preserving the relay-unreadable packet portion without decryption and without requiring readable access to the protected responder metadata or the encrypted responder-status payload;
the authorized incident command gateway comprising a processor and memory storing or accessing one or more responder authorization records, responder-context records, or incident records;
wherein the authorized incident command gateway is configured to receive the encrypted responder-status LoRa packet, access or resolve the protected responder metadata from the relay-unreadable packet portion, validate packet information associated with the encrypted responder-status LoRa packet and the accessed or resolved protected responder metadata against at least one responder authorization record, responder-context record, or incident record before displaying or routing decrypted responder-status data, prevent display or downstream routing of the encrypted responder-status LoRa packet when the validation is unsuccessful, and, when the validation is successful, decrypt the encrypted responder-status payload, associate decrypted responder-status data with a validated responder-context record or incident record, and generate or update an incident command display showing at least one responder-status condition.

18. The system of claim 17, wherein the responder-carried LoRa module is configured to classify the responder-status data as routine, urgent, or emergency based on at least one of oxygen-status data, no-motion state, fall state, temperature exposure state, vital-sign state, battery state, environmental state, manual distress data, or Mayday data, and to assign a packet priority based on the classification.

19. The system of claim 17, wherein the authorized incident command gateway is configured to determine or update a responder location, last-known location, or movement path using at least one of received signal strength, anchor-node proximity, vehicle-gateway proximity, perimeter-node proximity, hop-path data, inertial data, barometric data, floor-plan data, building-map data, or manual confirmation.

20. The system of claim 17, wherein the authorized incident command gateway is configured to generate a responder packet lifecycle audit record linking the encrypted responder-status LoRa packet, responder-carried LoRa module, responder identifier, protected responder metadata accessed or resolved by the authorized incident command gateway, responder-context record or incident record, validation result, decrypted responder-status data, displayed responder-status condition, downstream routing action, and timestamp.
